SEQUENCE LISTING (32 sequences)

SEQ ID NO: 1 - DNA, 22 bp, Artificial
  Primer for URA3 delete
ggagaatccatacaagaaatcg

SEQ ID NO: 2 - DNA, 26 bp, Artificial
  Primer for URA3 delta r
gtaaaatactgttacttggttctggc

SEQ ID NO: 3 - DNA, 24 bp, Artificial
  Primer HIS3-Green U
caggcaagataaacgaaggcaaag

SEQ ID NO: 4 - DNA, 25 bp, Artificial
  Primer HIS3-Green R
cactacggtgatgatcattcttgcc

SEQ ID NO: 5 - DNA, 82 bp, Artificial
  Primer URA3 fragment
ggcaagaatgatcatcaccgtagtgatcattctatacgtgtcattctgaacgaggcgcgctttccgattcggtaatctccga

SEQ ID NO: 6 - DNA, 60 bp, Artificial
  Primer HIS3-40Uc
gcattaccttgtcatcttcagtatcatactgttcgtatacgggtaataactgatataatt

SEQ ID NO: 7 - DNA, 27 bp, Artificial
  Primer TRP1-988
ctattagctgaattgccactgctatcg

SEQ ID NO: 8 - DNA, 21 bp, Artificial
  Primer TRP1-28r
gctgcctttgtgtgcttaatc

SEQ ID NO: 9 - DNA, 78 bp, Artificial
  Primer TRP1-URA3
gattaagcacacaaaggcagcgcaggccttttgaaaagcaagcataaaagatctaaacatagattcggtaatctccga

SEQ ID NO: 10 - DNA, 60 bp, Artificial
  Primer TRP1-40r
aggcaagtgcacaaacaatacttaaataaatactactcaggggtaataactgatataatt

SEQ ID NO: 11 - DNA, 20 bp, Artificial
  Primer LEU2-UP 3rd
gtctgcccctaagaagatcg

SEQ ID NO: 12 - DNA, 21 bp, Artificial
  Primer LEU2-down 3rd
cggatgcaaagttacatggtc

SEQ ID NO: 13 - DNA, 78 bp, Artificial
  Primer LEU2-URA3 3rd
gaccatgtaactttgcatccggacaaggaggagggcaccacacaaaaagttaggtgtaacagattcggtaatctccga

SEQ ID NO: 14 - DNA, 60 bp, Artificial
  Primer LEU2-40r
aatattaatgttaaagtgcaattctttttccttatcacgtgggtaataactgatataatt

SEQ ID NO: 15 - DNA, 37 bp, Artificial
  Primer EG NheI Fw
ctagctagcatgcaactgttcaatttgccattgaaag

SEQ ID NO: 16 - DNA, 44 bp, Artificial
  Primer EG XmaI Rev
tccccccgggtttgattatgttctttctatttgaatgagatatg

SEQ ID NO: 17 - DNA, 30 bp, Artificial
  Primer 1 for PGK promoter
ttttctcgagaaagatgccgatttgggcgc

SEQ ID NO: 18 - DNA, 30 bp, Artificial
  Primer 2 for PGK promoter
gcccgctagcgttttatatttgttgtaaaa

SEQ ID NO: 19 - DNA, 30 bp, Artificial
  Primer 1 for PGK terminater
gcccagatctgaaataaattgaattgaatt

SEQ ID NO: 20 - DNA, 30 bp, Artificial
  Primer 2 for PGK terminater
ttttgcggccgcagctttaacgaacgcaga

SEQ ID NO: 21 - DNA, 52 bp, Artificial
  Primer 1 for multi-cloning site
ttttgctagcgtcgacactagtggatcccccgggtctagagaattcagatct

SEQ ID NO: 22 - DNA, 52 bp, Artificial
  Primer 2 for multi-cloning site
ttttagatctgaattctctagacccgggggatccactagtgtcgacgctagc

SEQ ID NO: 23 - DNA, 42 bp, Artificial
  Primer 1 for GAPDH terminater
aaggaaaaaagcggccgcaccagttctcacacggaacaccac

SEQ ID NO: 24 - DNA, 51 bp, Artificial
  Primer 2 for GAPDH terminater
aaggaaaaaagcggccgctcaatcaatgaatcgaaaatgtcattaaaatag

SEQ ID NO: 25 - DNA, 40 bp, Artificial
  bgl1 primer 1
gatctccatggctgatgaactggcgttctctcctcctttc

SEQ ID NO: 26 - DNA, 40 bp, Artificial
  bgl1 primer 2
tggcgctcgagccttgcaccttcgggagcgccgcgtgaag

SEQ ID NO: 27 - DNA, 40 bp, Artificial
  Primer XYL2c-XhoI(F)
ccgctcgagaccagttctcacacggaacaccactaatgga

SEQ ID NO: 28 - DNA, 45 bp, Artificial
  Primer XYL2c-NotI(R)
atagtttagcggccgctcaatcaatgaatcgaaaatgtcattaaa

SEQ ID NO: 29 - DNA, 111 bp, Artificial
  Primer ss EGII forward
tcccccgggatgcaactgttcaatttgccattgaaagtttcattctttctcgtcctctcttacttttctttgctcgtttctcagcagactgtctggggccagtgtggaggt

SEQ ID NO: 30 - DNA, 40 bp, Artificial
  Primer ss EGII reverse
tcccccgggctactttcttgcgagacacgagctgaccaag

SEQ ID NO: 31 - DNA, 110 bp, Artificial
  Primer ss CBH2 forward
tcccccgggatgcaactgttcaatttgccattgaaagtttcattctttctcgtcctctcttacttttctttgctcgtttctcaagcttgctcaagcgtctggggccaatg

SEQ ID NO: 32 - DNA, 46 bp, Artificial
  Primer ss CBH2 reverse
tcccccgggttacaggaacgatgggtttgcgtttgtgagaagctgc

(19) United States
(12) Patent Application Publication
(10) Pub. No.: US 2012/0282666 A1
(43) Pub. Date: Nov. 8, 2012

(54) METHOD FOR PRODUCING ETHANOL

(76) Inventors: Hideo Noda, Amagasaki-shi (JP); Shohei Kaneko, Hyogo (JP); Akihiko Kondo, Hyogo (JP)

(21) Appl. No.: 13/512,895

(22) PCT Filed: Jun. 30, 2010
(86) PCT No.: PCT/JP2010/061131

§ 371 Date: Jul. 23, 2012

(30) Foreign Application Priority Data
Dec. 1, 2009 (JP) 2009-273968

(51) Int. Cl. C12P 7/10; C08B 1/00

(52) U.S. Cl. 435/165; 536/56

(57) ABSTRACT

The present invention provides a method for producing ethanol, including: subjecting a cellulose-based material to a hot-water treatment and an ammonia treatment to obtain a fermentation substrate; and reacting the fermentation substrate with a yeast to produce ethanol. The present invention also provides a method for producing a fermentation substrate for ethanol production by a yeast, including: subjecting a cellulose material to a hot-water treatment and an ammonia treatment to obtain a fermentation substrate. Thus obtained fermentation substrate can enhance the ethanol production by yeast.

### TECHNICAL FIELD

The present invention relates to ethanol production by yeast.

### BACKGROUND ART

In recent years, increased biofuel production from edible cereal grain (such as corn, potato, and sugar cane) has led to high food prices. It has thus became an urgent matter to produce ethanol from non-edible carbon source soft biomass (such as rice straw, straw, bagasse, rice husk, cotton, bamboo, paper, corn stover, and like herbaceous wastes).

It has been proposed to apply a biomass to acidic treatment or supercritical treatment to obtain glucose, which can be utilized by fermentation microorganisms, from biomass containing cellulose or hemicellulose.

Conventional methods for producing glucose using a cellulosic substance as a source material include acidic saccharification and enzymatic saccharification. It has been known as acidic treatment that saccharification with dilute acid, that is, a cellulose-based material is saccharified by treating the material with a dilute acid at high temperatures (200° C. or higher), and saccharification of a cellulosic substance with concentrated sulfuric acid or the like. However, in either of the method, a cellulosic substance is hydrolyzed under severe conditions, so that the secondary degradation reaction of glucose, which is a hydrolysis product of a cellulosic substance, occurs. Accordingly, the saccharification rate is as poor as about 50%, and there is a need to remove such a degradation product of glucose from the saccharified solution. It has several problems that a saccharified solution failing to remove the degradation product of glucose is used as a carbon source for fermentation.

The enzymatic saccharification allows saccharification of a cellulosic substance under mild conditions, but is problematic in that a long period of time is required for sufficient saccharification due to a low rate of saccharification reaction; and large amounts of enzyme is required for sufficient saccharification due to poor potencies of commercially available enzymes for use in saccharification, leading to increases in the cost of enzymes.

It has been attempted to attain ethanol fermentation directly from non-edible carbon sources by modifying a fermentation microorganism originally incapable of utilizing principal components of soft biomass, such as cellulose and hemicellulose, with bioengineering techniques. As such bioengineering techniques, cell surface-displaying techniques are suitably used. For example, yeasts have been engineered to surface-display a group of cellulolytic enzymes with cell surface-displaying techniques (Patent Documents 1 and 2). Although the yeast Saccharomyces cervisiae is incapable of metabolizing xylose, It has been attempted to produce ethanol from xylan found in birches using a engineered Saccharomyces cervisiae to surface-display xylan-degrading enzymes xylanase 2 (XYNII) derived from Trichoderma reesei and β-xylosidase (XylA) derived from Aspergillus oryzae and express genes for xylose reductase (XR) and xylitol dehydrogenase (XDH) (both derived from Pichia stipitis) and xylulokinase (XK) (derived from Saccharomyces cervisiae) (Non-Patent Document 1).

A yeast that surface-displays cellulase has been used concomitantly with a cellulase enzyme during fermentation for producing ethanol from a soft-biomass substrate, in order to increase the produced amount of ethanol. In terms of cost, it is desirable to reduce the amount of concomitant enzyme. Also, it is also desirable to create such an environment that a substrate degrading enzyme can act on a soft-biomass substrate more effectively to produce ethanol more efficiently.

It has been known, for example, as the pre-treatment for ethanol production from corn stover, that hydrolytic production of sugar by the treatment including hydrothermal treatment (such as liquid hot-water treatment, steam explosion treatment, or hot-compressed water treatment (Patent Document 1)) as well as ammonia explosion (AFEX), ammonia recycle percolation (ARP), lime treatment, pH control, and dilute acid treatment (Non-Patent Documents 2 and 3).

Since a soft biomass contains lignin, as well as cellulose and hemicellulose, it is desirable to remove lignin to facilitate the ethanol production reaction. Many papers have been reported on preferential degradation of lignin by ammonia treatment (Non-Patent Documents 4 to 7).

### PRIOR ART DOCUMENTS

### Patent Documents

Patent Document 1: WO 01/79483

Patent Document 2: Japanese Laid-Open Patent Publication No. 2008-86310

### Non-Patent Documents

Non-Patent Document 1: S. Katahira et al., Applied and Environmental Microbiology, 2004, vol. 70, pp. 5407-5414

Non-Patent Document 2: Mosier et al., Bioresour. Technol., 2005, vol. 96(6), pp. 673-686

Non-Patent Document 3: Wyman et al., Bioresour. Technol., 2005, vol. 96(18), pp. 2026-2032

Non-Patent Document 4: Y Kim et al., Bioresource Technology, 2008, vol. 99, pp. 5206-5215

Non-Patent Document 5: T. H. Kim et al., Bioresource Technology, 2005, vol. 96, pp. 2007-2013

Non-Patent Document 6: T. H. Kim et al., Applied Biochemistry and Biotechnology, 2007, vol. 136, pp. 136-140

Non-Patent Document 7: T. H. Kim et al., Bioresource Technology, 2003, vol. 90, pp. 39-47

Non-Patent Document 8: Appl. Microbiol. Biotech., 2002, vol. 60, pp. 469-474

Non-Patent Document 9: Applied and Environmental Microbiology, 2002, vol. 68, pp. 4517-4522

Non-Patent Document 10: R. Akada et al., Yeast, 2006, vol. 23, pp. 399-405

Non-Patent Document 11: Rose et al., Methods in Yeast genetics, A Laboratory Course Manual, Cold Spring Harbor Laboratory Press: Cold Spring Harbor, N.Y. 1990

Non-Patent Document 12: P. N. Lipke et al., Mol. Cell. Biol., August, 1989, 9(8), pp. 3155-65

Non-Patent Document 13: Y. Fujita et al., Applied and Environmental Microbiology, 2002, vol. 68, pp. 5136-41

Non-Patent Document 14: Y. Fujita et al., Applied and Environmental Microbiology, 2004, vol. 70, pp. 1207-12

Non-Patent Document 15: Takahashi et al., Appl. Microbiol. Biotechnol., 2001, vol. 55, pp. 454-462

### SUMMARY OF INVENTION

### Problems to be Solved by the Invention

An object of the present invention is to provide an efficient method for producing ethanol using soft biomass.

### Means for Solving the Problems

The present invention provides a method for producing ethanol, comprising:

subjecting a cellulose-based material to a hot-water treatment and an ammonia treatment to obtain a fermentation substrate; and

reacting the fermentation substrate with a yeast to produce ethanol.

In an embodiment, the cellulose-based material is subjected to the hot-water treatment followed by the ammonia treatment.

In an embodiment, the yeast is a cellulase yeast.

In a further embodiment, the cellulase yeast is a cellulase surface-displaying yeast that displays endoglucanase, cellobiohydrolase, and β-glucosidase on the cell surface.

In an embodiment, the reacting is carried out by fed-batch addition of the fermentation substrate.

The present invention also provides a method for producing a fermentation substrate for ethanol production by a yeast, comprising:

subjecting a cellulose material to a hot-water treatment and an ammonia treatment to obtain a fermentation substrate.

### Effects of Invention

According to the present invention, a method for efficiently producing ethanol from a cellulose-based material is provided.

### BRIEF DESCRIPTION OF DRAWINGS

FIG. 1 is a graph showing the time course of the production of ethanol by the cellulase surface-displaying yeast or the wild-type yeast with addition of cellulase in fed-batch fermentation using the hot-water-treated rice straw.

FIG. 2 is a graph showing the time course of the generation of phenolic compounds by the cellulase surface-displaying yeast or the wild-type yeast with addition of cellulase in fed-batch fermentation using the hot-water-treated rice straw.

FIG. 3 is a graph showing the time course of the production of ethanol by the cellulase surface-displaying yeast or the wild-type yeast with addition of cellulase in fed-batch fermentation using the hot-water-treated rice straw, the ammonia-treated rice straw, or the rice straw subjected to the hot-water treatment followed by the ammonia treatment.

FIG. 4 is a graph showing the time course of the production of ethanol by the cellulase surface-displaying yeast, the cellulase secreting yeast, or the wild-type yeast with addition of cellulase in fed-batch fermentation using the hot-water-treated DDG.

FIG. 5 is a graph showing the time course of the production of ethanol by the cellulase surface-displaying yeast, the cellulase secreting yeast, or the wild-type yeast with addition of cellulase in fed-batch fermentation using the ammonia-treated DDG.

FIG. 6 is a graph showing the time course of the production of ethanol by the cellulase surface-displaying yeast, the cellulase secreting yeast, or the wild-type yeast with addition of cellulase in fed-batch fermentation using the DDG subjected to the hot-water treatment followed by the ammonia treatment.

FIG. 7 is a graph showing the time course of the production of ethanol by the cellulase surface-displaying yeast or the wild-type yeast with addition of cellulase in fed-batch fermentation using the DDG subjected to hot-water treatment followed by ammonia treatment or the DDG subjected to ammonia treatment followed by hot-water treatment.

### MODE FOR CARRYING OUT THE INVENTION

(Cellulase Yeast)

The “cellulase yeast” herein refers to any yeast capable of hydrolyzing cellulose and producing ethanol from glucose. An yeast (such as a wild-type yeast) originally having no or little cellulose hydrolysis ability (herein, which may be also referred to as a “noncellulolytic yeast”) can be genetically engineered to express at least one cellulolytic enzymes (described in detail below), thereby giving a transformed yeast in which a cellulose hydrolysis ability is provided or enhanced, which is encompassed in the “cellulase yeast”. The “cellulase yeast” is preferably the cellulase yeast prepared as set forth below, but is not limited thereto.

The cellulolytic enzymes may be derived from any cellulolytic enzyme-producing microorganisms. Typical examples of cellulolytic enzyme-producing microorganisms include those belonging to the genus Aspergillus (for example, Aspergillus aculeatus, Aspergillus niger, and Aspergillus oryzae), the genus Trichoderma (for example, Trichoderma reese, the genus Clostridium (for example, Clostridium thermocellum), the genus Cellulomonas (for example, Cellulomonas fimi and Cellulomonas uda), the genus Pseudomonas (for example, Pseudomonas fluorescence), and the like.

The cellulolytic enzymes may be enzymes capable of cleaving β1,4-glucosidic linkage. Typical examples of enzymes capable of cleaving β1,4-glucosidic linkage include, but are not limited to, endo-β1,4-glucanase (hereinafter simply referred to as “endoglucanase”), cellobiohydrolase, and β-glucosidase.

Endoglucanase is usually referred to also as cellulase and can cleave cellulose intramolecularly (“intramolecular cellulose cleaving”) to generate glucose, cellobiose, and cello-oligosaccharide (the degree of polymerization may be 3 or greater and usually 10 or less but it is not limited thereto). Endoglucanase is highly reactive toward cellulose having a low degree of crystallization or noncrystalline cellulose such as amorphous cellulose, soluble cello-oligosaccharide, and carboxymethylcellulose (CMC) and other cellulose derivatives, but is poorly reactive toward cellulose microfibril, which has a crystalline structure. Endoglucanase is a typical example of an enzyme capable of hydrolyzing noncrystalline cellulose (hereinafter also referred to as a “noncrystalline cellulose hydrolyzable enzyme”). There are five kinds of endoglucanase, which are referred to as endoglucanase I (EGI), endoglucanase II (EGII), endoglucanase III (EGIII), endoglucanase IV (EGIV), and endoglucanase V (EGV), respectively, and distinguished by the difference in amino acid sequence but have in common an action of intramolecular cellulose cleaving. For example, endoglucanase derived from Trichoderma reesei (especially, EGII) may be used, but endoglucanase is not limited thereto.

Cellobiohydrolase can degrade cellulose from either the reducing terminal or the nonreducing terminal thereof to release cellobiose (“cellulose molecule terminal cleaving”). Cellobiohydrolase can degrade crystalline cellulose such as cellulose microfibril, which has a crystalline structure, but is poorly reactive toward cellulose having a low degree of crystallization or noncrystalline cellulose such as carboxymethylcellulose (CMC) and other cellulose derivatives. Cellobiohydrolase is a typical example of an enzyme capable of hydrolyzing crystalline cellulose (hereinafter also referred to as a “crystalline cellulose hydrolyzable enzyme”). Due to the rigid structure of crystalline cellulose with the dense intermolecular and intramolecular hydrogen bonding, the hydrolysis of crystalline cellulose by cellobiohydrolase may be slower than the hydrolysis of noncrystalline cellulose by endoglucanase. There are two kinds of cellobiohydrolase, which are referred to as cellobiohydrolase 1 (CBH1) and cellobiohydrolase 2 (CBH2), respectively, and distinguished by the difference in amino acid sequence but have in common an action of cellulose molecule terminal cleaving. For example, cellobiohydrolase derived from Trichoderma reesei (especially, CBH2) may be used, but cellobiohydrolase is not limited thereto.

β-Glucosidase is an exo-hydrolase, which releases glucose units from the nonreducing end in cellulose. β-Glucosidase can cleave the β1,4-glucosidic linkage between aglycon or a sugar chain and β-D-glucose, and hydrolyze cellobiose or cello-oligosaccharide to generate glucose. β-Glucosidase is a typical example of an enzyme capable of hydrolyzing cellobiose or cello-oligosaccharide. There is currently one kind of β-glucosidase known, which is called β-glucosidase 1 (BGL1). For example, β-glucosidase derived from Aspergillus aculeatus (especially, BGL1) may be used, but β-glucosidase is not limited thereto.

As described in detail below, for example, a cellulase yeast may be produced by introducing a group of genes for cellulolytic enzymes into a noncellulolytic yeast (such as a wild-type yeast). The group of genes for cellulolytic enzymes includes a gene for an enzyme capable of hydrolyzing crystalline cellulose and a gene for an enzyme capable of hydrolyzing noncrystalline cellulose. The enzyme capable of hydrolyzing crystalline cellulose (“crystalline cellulose hydrolyzable enzyme”) refers to any enzyme that can hydrolyze cellulose that has a crystalline structure such as microfibril, and an example may be, but is not limited to, cellobiohydrolase. The enzyme capable of hydrolyzing noncrystalline cellulose (“noncrystalline cellulose hydrolyzable enzyme”) refers to any enzyme that can hydrolyze the chain of cellulose having a low degree of crystallization or of noncrystalline cellulose such as amorphous cellulose although not hydrolyze cellulose having a crystalline structure, and an example may be, but is not limited to, endoglucanase. Preferably, the group of genes for enzymes capable of hydrolyzing cellulose further includes a gene for an enzyme capable of hydrolyzing cellobiose or cello-oligosaccharide. Cello-oligosaccharide is as described above. An example of an enzyme capable of hydrolyzing cellobiose or cello-oligosaccharide may be, but is not limited to, β-glucosidase.

For example, a cellulase yeast may be produced by genetically modifying a noncellulolytic yeast (such as a wild-type yeast) with genes for a crystalline cellulose hydrolyzable enzyme and/or a noncrystalline cellulose hydrolyzable enzyme. The genetically modifying can be preferably made so that expression levels of both a crystalline cellulose hydrolyzable enzyme and a noncrystalline cellulose hydrolyzable enzyme are increased. That is, increased integration copy numbers of both the genes for the respective enzymes are introduced into a noncellulolytic yeast to give a transformed yeast. The manner of expression of the crystalline cellulose hydrolyzable enzyme and the noncrystalline cellulose hydrolyzable enzyme is not limited as long as the expressed enzymes act on a cellulosic substrate. For example, the manner of expression may be surface display or secretory expression. At least one or both of the crystalline cellulose hydrolyzable enzyme and the noncrystalline cellulose hydrolyzable enzyme may be either surface-displayed or secreted. An yeast may be transformed such that the surface display and the secretion of the crystalline cellulose hydrolyzable enzyme and the noncrystalline cellulose hydrolyzable enzyme occur together.

It is preferable that the gene for an enzyme capable of hydrolyzing cellobiose or cello-oligosaccharide is integrated in the cellulase yeast, which may enhance the glucose production from cellulose. This enzyme may also be surface-displayed or secreted, and preferably it is surface-displayed. To more efficiently perform ethanol fermentation from cellulose, it is preferable that an enzyme capable of hydrolyzing cellobiose or cello-oligosaccharide is also expressed on the yeast.

For the cellulase yeast, the integration copy number of each of the gene for the crystalline cellulose hydrolyzable enzyme and the gene for the noncrystalline cellulose hydrolyzable enzyme may be at least two copies relative to one copy of the integration copy number of the gene for the enzyme capable of hydrolyzing cellobiose or cello-oligosaccharide.

In an example, cellobiohydrolase may be used as a crystalline cellulose hydrolyzable enzyme, and endoglucanase may be used as a noncrystalline cellulose hydrolyzable enzyme. To increase the expression of both enzymes, a single yeast may be transformed with at least two vectors which contain together expression cassettes (described in detail below) of genes for these enzymes. A single yeast may be transformed with at least two pairs of the combination of vectors each of which contains an expression cassette of one of genes for these enzymes. When an industrial yeast, which does not have any auxotrophic marker originally, is to be transformed, it is desirable to provide it with an auxotrophic marker, and it is thus preferable in terms of operational efficiency to prepare a vector (examples of vectors include those described in the examples below) containing an auxotrophic marker together with expression cassettes of the genes for these enzymes, as described in detail below.

The cellulase yeast may further contain β-glucosidase integrated as an enzyme capable of hydrolyzing cellobiose or cello-oligosaccharide.

In one embodiment, the cellulase yeast may be a yeast expressing the three enzymes of β-glucosidase, cellobiohydrolase, and endoglucanase. In a further embodiment, the cellulase yeast may be a yeast displaying these respective enzymes on the cell surface, which may also be referred to as a “cellulase surface-displaying yeast” for convenience.

The production of ethanol may be enhanced by increasing the integration copy numbers of cellobiohydrolase and endoglucanase relative to the integration copy number of β-glucosidase gene. Therefore, at least two copies of each of the genes for the cellobiohydrolase and the endoglucanase may be integrated relative to one copy of the integration copy number of the gene for the β-glucosidase. Three or more copies of each of the genes for the cellobiohydrolase and the endoglucanase may be integrated relative to one copy of the integration copy number of the gene for the β-glucosidase. By genetically modifying a noncellulolytic yeast (such as a wild-type yeast) in such a way, it is possible to obtain an yeast having increased production of ethanol.

In one embodiment, the genes are integrated such that at least one or both of the cellobiohydrolase and the endoglucanase are either surface-displayed or secreted and the β-glucosidase is surface-displayed. Preferably, the cellobiohydrolase, the endoglucanase, and the β-glucosidase may be surface-displayed.

Hereinafter, preparation of cellulase yeast will be described, but the present invention is not limited thereto.

The gene of an enzyme to be expressed can be obtained from a microorganism that produces the enzyme by PCR or hybridization with primers or a probe designed based on known sequence information.

The enzyme gene can be used to construct an expression cassette. The expression cassette may contain so-called regulatory factors such as a promoter and a terminator, and an enhancer that regulate the expression of the gene. The promoter and the terminator may be those of the gene to be expressed, or those derived from a different gene may be used. For the promoter and the terminator, promoters and terminators of GAPDH (glyceraldehyde 3′-phosphate dehydrogenase), PGK (phosphoglycerate kinase), GAP (glyceraldehyde 3′-phosphate), and like may be used, but the selection of a promoter and a terminator may depend on the expression of the enzyme gene of interest and they can be suitably selected by those skilled in the art. Additional factors that regulate the expression (such as an operator and an enhancer) or the like may be contained as necessary. Expression regulatory factors such as operators and enhancers may also be suitably selected by those skilled in the art. The expression cassette may further contain a necessary functional sequence depending on the purpose of the expression of the gene. The expression cassette may contain linkers as necessary

For the construction of the expression cassette containing the gene, a cell surface engineering technique may be used. Examples include, although they are not limited to, (a) displaying an enzyme on the cell surface via the GPI anchor of a cell surface-localized protein, (b) displaying an enzyme on the cell surface via the sugar chain binding domain of a cell surface-localized protein, and (c) displaying an enzyme on the cell surface via a periplasm protein (another receptor molecule or target receptor molecule). Relevant techniques for cell surface engineering are described also in, for example, Patent Documents 1 and 2.

Examples of usable cell surface-localized proteins include α- or a-agglutinin, which is an yeast flocculation protein (for use as the GPI anchor); Flo1 proteins (Flo1 proteins can be used as the GPI anchor with modification of amino acid length on the N-terminal; for example, Flo42, Flo102, Flo146, Flo318, Flo428, and the like; Non-Patent Document 8: Note that Flo1326 refers to the full-length Flo1 protein); Flo proteins (there are no GPI anchor functions and flocculability is used, Floshort or Flolong; Non-Patent Document 9); invertase, which is a periplasm-localized protein (no GPI anchor is used); and the like.

First, (a) use of GPI anchor is described. The gene coding for a protein localized on a cell surface by a GPI anchor has, in order from the N-terminal, a gene coding for a secretion signal sequence, a gene coding for a cell surface-localized protein (a sugar chain binding protein domain), and a gene coding for a GPI anchor attachment recognition signal sequence. A cell surface-localized protein (a sugar chain binding protein) expressed from this gene in a cell is directed outside the cell membrane by a secretion signal, and then a GPI anchor attachment recognition signal sequence binds to the GPI anchor of the cell membrane via a specifically truncated C-terminal portion to immobilize the protein on the cell membrane. Subsequently, the protein is cleaved near the root of the GPI anchor by PI-PLC, and integrated into the cell wall, and immobilized on the cell surface, resulting in display of the protein on the cell surface.

Here, the GPI anchor refers to a glycolipid having a basic structure of ethanolamine-phosphate-6-mannose-α1-2-mannose-α1-6-mannose-α1-4-glucosamine-α1-6-inositol-phospholipid called glycosyl phosphatidylinositol (GPI), and PI-PLC refers to phosphatidylinositol-dependent phospholipase C.

Here, the secretion signal sequence refers to an amino acid sequence rich in highly hydrophobic amino acids, generally linked to the N terminal of a protein to be secreted outside the cell, including the periplasm, i.e., secretory protein, and is usually eliminated when the secretory protein is secreted from inside the cell through the cell membrane to the outside the cell. Any secretion signal sequence may be used irrespective of its origin as long as the secretion signal sequence can direct the expression product to the cell membrane. For example, the secretion signal sequence of glucoamylase, the signal sequence of yeast α- or a-agglutinin, the secretion signal sequence of the expression product itself are suitably used for the secretion signal sequence. The secretion signal sequence and the pro-sequence may partially or entirely may remain in the N terminal without affecting the activity of a protein fused to cell surface binding proteins adversely.

The GPI anchor attachment recognition signal sequence is a sequence recognized upon the binding of the GPI anchor to a cell surface-localized protein and is usually located at or near the C-terminal of the cell surface-localized protein. For example, the sequence of the C-terminal portion of yeast α-agglutinin is suitably used for the GPI anchor attachment signal sequence. Since a GPI anchor attachment recognition signal sequence is contained in the C-terminal of the sequence of 320 amino acids from the C-terminal of α-agglutinin, a DNA sequence coding for the sequence of 320 amino acids from the C-terminal is particularly useful as a gene for use in the method.

Therefore, for example, in a sequence having a DNA coding for a secretion signal sequence—a structural gene coding for a cell surface-localized protein—a DNA sequence coding for a GPI anchor attachment recognition signal, the entire or a part of the sequence of the structural gene coding for a cell surface-localized protein can be replaced with a DNA sequence coding for an enzyme protein of interest so as to obtain a recombinant DNA for displaying the enzyme protein of interest on the cell surface via a GPI anchor. In case of the cell surface-localized protein is α-agglutinin, it is preferable to introduce a DNA coding for the enzyme of interest such that the sequence coding for the sequence of 320 amino acids from the C-terminal of the α-agglutinin is retained. For this purpose, the “3′ half region of α-agglutinin gene” may be used. Such a recombinant DNA can be introduced into an yeast for expression to display the enzyme on the cell surface, where the enzyme is immobilized on the surface via the C-terminal.

Next, (b) use of a sugar chain binding protein domain is described. The cell surface-localized protein can be a sugar chain binding protein, and the sugar chain binding protein domain thereof has a plurality of sugar chains which can interact or be entangled with sugar chains present in the cell wall to leave the protein on the cell surface. Examples include sugar chain binding sites of lectin, lectin-like proteins, and the like. Typical examples include the flocculation functional domain of a GPI anchor protein and the flocculation functional domain of a FLO protein. The flocculation functional domain of a GPI anchor protein refers to a domain that is located on the side of N-terminal relative to the GPI anchoring domain, has a plurality of sugar chains, and is thought to be involved in flocculation.

The linkage of sugar chain binding protein domain (or flocculation functional domain) of a cell-surface localized protein with an enzyme protein of interest allows the enzyme to be displayed on the cell surface. Depending on the enzyme of interest, the enzyme may be linked (1) on the side of N-terminal or (2) on the side of C-terminal of the sugar chain binding protein domain (or flocculation functional domain) of a cell surface-localized protein, or the same or different enzymes may be linked (3) on both sides of N-terminal and C-terminal. In the invention, (1) a DNA coding for a secretion signal sequence—a gene coding for an enzyme of interest—a structural gene coding for the sugar chain binding protein domain (or flocculation functional domain) of a cell surface-localized protein; or (2) a DNA coding for a secretion signal sequence—a structural gene coding for the sugar chain binding protein domain (or flocculation functional domain) of a cell surface-localized protein—a gene coding for an enzyme of interest; or (3) a DNA coding for a secretion signal sequence—a gene coding for a first enzyme of interest—a structural gene coding for the sugar chain binding protein domain (or flocculation functional domain) of a cell surface-localized protein—a gene coding for a second enzyme of interest (the first and second enzymes may be same or different) may be produced to obtain a recombinant DNA for displaying the enzyme(s) of interest on the cell surface. Using the flocculation functional domain, the DNA sequence coding for a GPI anchor attachment recognition signal sequence may be partially present or may not be present in the recombinant DNA since the GPI anchor is not involved in cell surface display. The use of the flocculation functional domain is very advantageous in that: the enzyme can be displayed on the cell surface in a more suitable length because the length of the domain can be easily modified (for example, any of Floshort and Flolong can be selected); and the enzyme can be linked on either side of the N-terminal or the C-terminal.

Next, (c) use of a periplasm protein (another receptor molecule or target receptor molecule) is described. This method is based on the fact that an enzyme protein of interest can be expressed on the cell surface as a fused protein with the periplasm protein. An example of the periplasm protein may be invertase (Suc2 protein). The enzyme of interest may be suitably fused on the side of N-terminal or C-terminal depending on the periplasm protein.

A method for expression of an enzyme to secrete it outside an yeast cell is well known to those skilled in the art. A recombinant DNA in which the structural gene of the enzyme of interest is linked to a DNA coding for the secretion signal sequence may be prepared and introduced into an yeast.

A method for expression of a gene in an yeast cell is also well known to those skilled in the art. In this case, a recombinant gene to which the structural gene of interest is linked without using the cell surface display factor or the secretion signal as described above may be prepared and introduced into an yeast.

The synthesis and the linkage of DNA including various sequences may be performed using techniques commonly used by those skilled in the art. For example, the linkage of the secretion signal sequence and the structural gene for the enzyme of interest can be carried out using site-directed mutagenesis technique, thereby allowing accurate cleavage of secretion signal sequence and active expression of enzyme.

An enzyme gene or an expression cassette may be inserted into a vector in a plasmid form. It is preferable that the vector is an yeast—E. coli shuttle vector for facilitating the procedure for obtain a DNA. The vector may contain a regulatory sequence as described above, as necessary. It is further preferable that, for example, the starting material of vector preparation has an origin of replication (Ori) of a 2 μm plasmid for yeast and an origin of replication of a plasmid ColE1 for E. coli as well as an yeast selectable marker (examples include drug-resistant genes and auxotrophic marker genes (for example, genes coding for imidazoleglycerol phosphate dehydrogenase (HIS3), beta-isopropylmalate dehydrogenase (LEU2), tryptophan synthase (TRPS), argininosuccinase lyase (ARG4), N-(5′-phosphoribosyl)anthranilic acid isomerase (TRP1), histidinol dehydrogenase (HIS4), orotidine-5-phosphate decarboxylase (URA3), dihydroorotic acid dehydrogenase (URA1), galactokinase (GAL1), and alpha-aminoadipic acid reductase (LYS2)) and an E. coli selectable marker (such as a drug resistant gene).

Examples of the starting plasmid include pYGA2270 or pYE22m, which contains a GAPDH promoter and a GAPDH terminator; pWI3, which contains UPR-ICL (an upstream region of isocitrate lyase) and Term-ICL (a terminator region of isocitrate lyase); pGK406, which contains a PGK promoter and a PGK terminator, and the like. The plasmid vector can be prepared as in Examples set forth below.

The “introduction” of a gene or a DNA herein means not only the introduction of a gene or a DNA into a cell but also the expression thereof. The “transformation” means that a gene or DNA is introduced into a cell to express so that the genetic trait of the host can be changed. For introduction or transformation into an yeast cell, specific examples include lithium acetate method, protoplast method, and the like for yeast cells. The DNA to be introduced may be present in a plasmid form or may be incorporated into a chromosome after being inserted into the gene of a host or through homologous recombination with the gene of a host.

The host yeast is a noncellulolytic yeast and this may be a wild-type yeast. The kind of yeast is not particularly limited, and in particular, yeasts that belong to the genus Saccharomyces are preferable, with Saccharomyces cerevisiae being preferable. Wild-type industrial yeasts are preferable. The wild-type yeast may be genetically modified to enhance an ability of alcoholic fermentation from a substrate monosaccharide (for example, glucose).

The term “industrial yeast” refers to any yeasts used conventionally in ethanol fermentation (for example, sake yeasts, shochu yeasts, wine yeasts, beer yeasts, baker's yeasts, and the like). Among industrial yeasts, sake yeasts are preferable in regard to high ethanol fermentability and high ethanol resistance and genetic stability. An “industrial yeast” has high ethanol resistance and preferably is viable at ethanol concentrations of 10% or greater. Moreover, it is preferable that it has acid resistance, heat resistance, and the like. More preferably, it may be flocculable. Examples of industrial yeast which has such properties include the strains Saccharomyces cerevisiae NBRC1440 (MATα, haploid yeast, heat resistant and acid resistant, flocculable) and NBRC1445 (MATa, haploid yeast, heat resistant and acid resistant, not flocculable) both available from the National Institute of Technology and Evaluation.

Since the industrial yeast has extremely strong ethanol resistance, it is possible to apply it directly to ethanol fermentation after monosaccharide production. In particular, the industrial yeast is preferable because it is resistant to any stresses under culturing and shows stable cell proliferation even in industrial production where a precise control of culturing conditions is difficult, which may result in severe culturing conditions. Since industrial yeasts form polyploids, it is possible to integrate a plurality of gene constructs (expression vectors) into homologous chromosomes, and as a result, the amount of the protein of interest expressed is higher compared to the integration into laboratory yeasts, which are often haploids.

Industrial yeasts are often prototrophs and lack an auxotrophic marker suitable for selecting for a transformant. Accordingly, a specific auxotrophic marker suitable for introducing a gene of interest is provided with an industrial yeast, especially, into an yeast lacking an auxotrophy and highly resistant to ethanol (preferably viable at ethanol concentrations of 10% or greater) to facilitate the introduction of the gene of interest thereinto. Examples of the auxotrophic marker include, but not limited to, uracil auxotrophy, trypsin auxotrophy, leucine auxotrophy, histidine auxotrophy, and the like, in view of the applicability in gene manipulation. To provide uracil auxotrophy, a normal ura3 gene of industrial yeast can be replaced with an ura3⁻ fragment obtained from an uracil auxotrophic mutant (for example, Saccharomyces cerevisiae MT-8). To provide an auxotrophy other than uracil auxotrophy (for example, trypsin auxotrophy, leucine auxotrophy, histidine auxotrophy, or the like), it is possible to design a fragment so as to adopt as the target and disrupt the gene thereof, for example, according to the method described in Non-Patent Document 10.

The industrial yeast into which the expression cassette as mentioned above has been introduced may be selected with an yeast selectable marker (for example, an auxotrophic marker mentioned above) as described above, and confirmed by determining the activity of the expressed enzyme protein. The immobilization of protein on cell surface may be confirmed by immunological antibody method using an anti-protein antibody and an FITC-labeled anti-IgG antibody.

(Ethanol Production)

Herein, the process of reacting a yeast with a substrate to produce ethanol is also referred to as a “fermentation process” for convenience.

According to the present invention, a cellulose-based material as described below is subjected to a hot-water treatment and an ammonia treatment prior to reacting with a yeast (“fermentation process”). A hot-water treatment may be followed by an ammonia treatment (which may also be referred to as “a hot-water treatment followed by an ammonia treatment” for convenience), or an ammonia treatment may be followed by a hot-water treatment (which may also be referred to as “an ammonia treatment followed by a hot-water treatment” for convenience).

A cellulose-based material subjected to a hot-water treatment and an ammonia treatment (preferably, subjected to a hot-water treatment followed by an ammonia treatment) is used as a substrate for the fermentation process. Herein, the substrate for the fermentation process is also referred to as a “fermentation substrate.”

The term “cellulose-based material” as used herein refers to any material, product, or composition which contains cellulose. The term “cellulose” refers to a fibrous polymer in which glucopyranoses are connected by a β1,4-glycosidic bond and includes derivatives and salts thereof as well as those that have a degree of polymerization reduced by degradation.

Within the “cellulose-based material”, any material containing cellulose, including, for example, paper waste generated in production or recycling of paper, cotton products such as old clothes and waste towels, and the xylem of trees or the leaves, stems, bark, and the like (especially, non-edible portions) of herbaceous plants that are not agriculturally harvested or are disposed of in food production) is encompassed. Within the “cellulose-based material”, cellulosic compounds such as carboxymethylcellulose (CMC), which is carboxymethylated cellulose, phosphoric acid-swollen cellulose, crystalline cellulose (for example, Avicel) and the like are also encompassed. Among the cellulosic compounds, phosphoric acid-swollen cellulose is suitably used as an alternative substrate of actual biomass cellulose to measure the cellololytic ability of cellulolytic enzyme.

The above illustrated cellulose-containing materials (especially, the xylem of trees and the leaves, stems and bark of herbaceous plants) may contain plant cell wall components, one of the principal components of which is cellulose. Plant cell walls usually contain cellulose, as well as hemicellulose and lignin, as their components. Depending on the plant species (especially, whether woody or herbaceous), the portion (organ, tissue) of plant, the extent of plant growth, or the like, the contents of such components may vary, but any portion of plant of any species at any growth stage may be used as long as it contains cellulose.

Therefore, cellulose-based materials also include any material, waste, and product containing the plant cell wall components as mentioned above. Insoluble dietary fiber is also encompassed within the “plant cell wall component-containing materials ” Cellulose-based materials include, in addition to the xylem of trees and the leaves, stems and bark of herbaceous plants as mentioned above, products processed from such portions, but use of discarded waste is preferable in view of reuse.

Examples of cellulose-based materials include cellulose compounds themselves and cellulose compound-containing compositions, and also rice husk, bamboo, bagasse, straw (such as rice, wheat, and barley straws), corncob, and other agricultural wastes, wooden materials (wood chip, scrap wood), old newspaper, magazine, cardboard, waste office paper, linters, cotton, pulp, waste pulp discharged from paper manufacturers, and the like. The present invention can be suitably used even with a cellulose-based material that contains lignin as the source material. Corn fiber or DDG (distiller's dried grain: a dried residue of corn distillation), which may be a by-product of the process of producing ethanol from corn, can also be used. Corn fiber is a husk portion of corn, which is removed prior to fermentation in the process of ethanol production. DDG is a solid residue after saccharification with enzyme and yeast fermentation, and is a portion (such as husk) of corn that excludes starch consumed in fermentation, and possibly includes the yeast used for fermentation. Corn fiber and DDG are both cellulose-containing materials (cellulose-based materials) Corn fiber and DDG are both available from corn starch producers or distributors (for example, Sungrain, Ltd.).

An example of the “hot-water treatment” may be a non-catalytic hydrothermal treatment as described in Patent Document 1. A cellulose-based material, ground as necessary, is mixed with water so as to attain a concentration of, for example, about 10% by weight, and this mixture is subjected to a treatment. According to the method described in Patent Document 1, for a batch process, although depending on the concentration to be treated, a cellulose-based material, for example when the concentration of the cellulose-based material is about 10% by weight, may be treated at 120 to 300° C., preferably 150 to 280° C., and more preferably 180 to 250° C., and generally the treatment time is preferably in the range of 1 hour to 15 seconds. For a continuous process, the temperature can be slightly higher owing to the time of thermal hysteresis, and, a cellulose-based material, for example when the concentration of the cellulose-based material is about 10% by weight, may be treated at 120 to 373° C. and preferably 150 to 320° C., preferably for 1 hour to 1 second. The temperature and time for a treatment may vary depending on the material used, and an increased temperature for the treatment may shorten the treatment time. As for application of pressure, such a pressure that a temperature within the aforementioned range can be reached may be set automatically by an apparatus or manually.

Examples of the “ammonia treatment” include treatment with ammonia solution (which may be also referred to as “ammonia solution treatment”), ammonia explosion (AFEX), and ammonia recycle percolation (ARP). For example, the treatment may be carried out as described in Non-Patent Documents 4 to 7 (preferably Non-Patent Document 5). The ammonia solution treatment may be carried out, for example, using aqueous ammonia at about 10 to about 25% by weight and preferably about 15% by weight, at about 100 to about 200° C., preferably about 150 to about 190° C., and more preferably about 170° C., for about 5 minutes to about 90 minutes. An example may be the ammonia treatment described in the Examples below, but the ammonia solution treatment is not particularly limited thereto. The conditions of treatment may be suitably set depending on the starting cellulose-based material. The ammonia treatment may be carried out only once or may be carried out repeatedly.

A hot-water treatment and an ammonia solution treatment may each be carried out in separate apparatuses, or may be carried out sequentially in the same apparatus. For example, a cellulose-based material that has been subject to a hot-water treatment or a cellulose-based material that has been subject to an ammonia solution treatment may be separated from the treatment liquid (solution) and then subjected to the subsequent treatment. Alternatively, a cellulose-based material may be subjected to a hot-water treatment and be directly placed under conditions for ammonia treatment (for example, after the hot-water treatment is completed, the treated material is left following changing the temperature inside the reactor to a temperature suitable for ammonia treatment, and injecting ammonia thereinto so as to attain a specific concentration), or conversely, a cellulose-based material may be subjected to an ammonia treatment, ammonia be removed, and then the treated cellulose-based material placed under conditions for hot-water treatment. After the ammonia treatment, ammonia may be removed and recovered by distillation or stripping, and the recovered ammonia can be used again for the treatment of another batch.

A hot-water treatment followed by an ammonia treatment (for example, a cellulose-based material that has been subjected to a hot-water treatment is subjected to an ammonia solution treatment) is preferable. Preferably, the hot-water treatment can be carried out at a high temperature (for example, 170 to 230° C.) for a specific time of period with sealing and under pressure as needed inside a reactor of an apparatus; the temperature inside the reactor can be then reduced to a lower temperature (for example, 150 to 190° C.); ammonia can be injected so as to attain a specific concentration; and the ammonia treatment can be carried out for a specific period of time. Thereafter, the temperature of liquid matter obtained after the hot-water treatment followed by ammonia treatment is lowered to, for example, 100° C. On a laboratory scale, ammonia can be evaporated from the liquid matter by leaving the reactor opened, and the remaining liquid matter can be then dried to further remove ammonia, and the resultant can be recovered as a fermentation substrate. On an industrial process scale, liquid matter having a lowered temperature of 100° C. can be introduced into a topper (atmospheric distillation tower) and refluxed so that, for example, 28 wt % of aqueous ammonia can be recovered from the top of the tower and the ammonia-free, treatment product can be taken as a fermentation substrate from the bottom of the tower. Possible ammonia content in this fermentation substrate is less than several ppm, which is at an acceptable level for industrial use of the fermentation substrate. In the hot-water treatment followed by ammonia treatment, the temperature during the respective processes of hot-water treatment, ammonia treatment, and ammonia removal may be lowered in series, thus making it possible to reduce any change in the temperature during a sequence of these processes. A larger amount of ethanol can be produced with the hot-water treatment followed by ammonia treatment.

The product after the hot-water treatment and the ammonia treatment may be suitably cooled or dried, and then used as a fermentation substrate. The procedure for drying is not particularly limited. Examples include freeze drying, low-temperature drying, ambient-temperature drying, high-temperature drying, and the like, and drying may be carried out under ventilation or under vacuum.

In the fermentation process, a cellulose-based material that has been subjected to a hot water treatment and an ammonia treatment as a fermentation substrate can be reacted with a yeast to produce ethanol.

In the fermentation process, a yeast capable of ethanol fermentation from glucose may be used, and a cellulase yeast may be suitably used. The cellulase yeast is as described above. While it is preferable that cellulose can be degraded into glucose with a single yeast, a combination of multiple different types of yeast may be used to degrade cellulose into glucose. The latter may be, for example, a combination of yeasts that express different enzymes so as to provide three enzymes of β-glucosidase, cellobiohydrolase, and endoglucanase in the fermentation process. It is preferable that β-glucosidase is surface-displayed. Also, it is preferable that cellobiohydrolase and endoglucanase are surface-displayed. In the fermentation process, a yeast that can be used for ordinary ethanol fermentation (for example, a wild-type industrical yeast) may be used alone or in combination with a cellulase yeast.

The reaction between a fermentation substrate and a yeast may be carried out by performing the culture (preferably, liquid culture) of the yeast in a medium containing the fermentation substrate. The fermentation process may be carried out under conditions for usually carrying out ethanol fermentation. The fermentation medium may further contain any component necessary or desirable for the growth of a yeast. The temperature during the reaction of the fermentation process may depend on the yeast used, and may usually be about 30° C. to about 38° C. The fermentation pH is preferably about 4 to about 6, and more preferably about 5. The culture for fermentation may be carried out anaerobically (the dissolved oxygen concentration may be, for example, about 1 ppm or less, more preferably about 0.1 ppm or less, and even more preferably about 0.05 ppm or less).

Examples of the manner of the fermentation process include batch, fed-batch, repeated batch, continuous, and the like, and any of these may be selected. Preferably, a fermentation substrate may be additionally input as needed during the fermentation process (fed-batch addition) to increase the produced amount of ethanol.

The input amount of yeast; the initial input amount as well as the amount and time point for additional input as necessary of fermentation substrate; and the fermentation period may be suitably determined depending on the factors including the type and state of substrate, the volume of fermentation culture, and the intended amount of fermentation ethanol to be produced. The amount and time point for additional input of fermentation substrate may be determined with monitoring the viscosity of the medium for fermentation, the produced amount of ethanol, or the generated amount of carbon dioxide during the process of fermentation.

The yeast may be cultured under aerobic conditions to increase the number of cells prior to the fermentation process. The medium may be selective or non-selective. The pH of the medium during culturing is preferably about 4 to about 6, and more preferably about 5. The dissolved oxygen concentration in the medium during culturing under aerobic conditions is preferably about 0.5 to about 6 ppm, more preferably about 1 to about 4 ppm, and even more preferably about 2 ppm. The temperature during culturing may be about 20 to about 45° C., preferably about 25 to about 40° C., and more preferably about 30 to about 37° C. The period of culturing may be determined based on the yeast load to be used in the fermentation reaction. For example, when culturing is carried out until the cell concentration of total yeast reaches 20 g (wet weight)/L or greater, more preferably 50 g (wet weight)/L or greater, and even more preferably 75 g (wet weight)/L or greater, the period of culturing may be about 20 to about 50 hours.

A cellulase enzyme may be used supplementally in the fermentation process. The “cellulase enzyme” includes a cellulase enzyme of any form that is isolated as an enzyme. Examples of the “cellulase enzyme” include an enzyme isolated and purified from a microorganism that produces cellulase (i.e., endoglucanase) as described above and an enzyme produced by genetic modification using a cellulase gene. Commercially available cellulase enzymes are also usable. An example of a commercially available cellulase enzyme is Cellulase SS, Trichoderma reesei-derived cellulase, a titer of 7.6 FPU/mL (“FPU” is an abbreviation for “Filter Paper Unit”, with “1 FPU” being the amount of enzyme that generates 1 μmol of reducing sugars corresponding to glucose from filter paper in 1 minute). In particular, when ethanol is industrially produced, a cellulase enzyme may be further added in the reaction of a cellulase yeast and a cellulose-based material to enhance production efficiency.

Since the conditions of ethanol fermentation vary during the process of fermentation, it is preferable to control the conditions to be within a specific range. The time course of fermentation may be monitored by, for example, gas chromatography, HPLC, or a like means commonly used by those skilled in the art.

After the fermentation process is completed, the medium, which contains ethanol, is removed from a fermenter, and is subjected to any separation process usually used by those skilled in the art, such as separating with centrifuge and distillation, to isolate ethanol.

Preferably, the yeast for reaction with the fermentation substrate (preferably a cellulase yeast, and more preferably a yeast that surface-displays any one or more (more preferably, all three) of three enzymes of β-glucosidase, cellobiohydrolase, and endoglucanase) and a cellulase enzyme if necessary are fixed to carriers, respectively, thus making reuse possible.

As the carrier and the method for immobilizing, carriers and methods commonly used by those skilled in the art may be used. Examples include carrier binding, entrapment, crosslinking, and the like.

A porous material is preferably used as the carrier. For example, preferable are polyvinyl alcohol, polyurethane foam, polystyrene foam, polyacrylamide, polyvinyl formal porous resin, silicone foam, and like foam and resin. The pore size of a porous material may be selected in consideration of the microorganism to be used and the size thereof. In the case of industrial yeast, the size is preferably 50 to 1000 μm.

The carrier may have any shape. In view of the strength of the carrier, culturing efficiency, and the like, the shape is preferably spherical or cubic. The size may be selected depending on the microorganism to be used, and it is generally preferable that the diameter is 2 to 50 mm where the carrier is spherical and one side has a length of 2 to 50 mm where the carrier is cubic.

According to the present invention, a fermentation substrate can be additionally input as needed during the fermentation reaction to increase the produced amount of ethanol. An increased viscosity of the fermentation medium after the input of substrate can be preferably reduced during the process of fermentation, thus facilitating to additionally input the fermentation substrate, making it possible to efficiently produce ethanol from the fermentation substrate, and making it easy to isolate ethanol after the fermentation process. Moreover, the supplementally added amount of cellulase enzyme can be reduced. Use of a cellulase yeast, and more preferably a yeast that surface-displays any one or more (more preferably, all three) of three enzymes of β-glucosidase, cellobiohydrolase, and endoglucanase can enhance these effects.

The present invention shall be described below by way of examples although the present invention is not limited to the examples.

### EXAMPLES

The strains Saccharomyces cerevisiae NBRC1440 (MATα) and Saccharomyces cerevisiae MT8-1 (MATa ade his3 leu2 trp1 ura3) used in the examples were obtained from the National Institute of Technology and Evaluation.

All PCR amplifications presented in the examples were performed using a KOD-Plus-DNA polymerase (Toyobo Co., Ltd.).

All Yeast transformations presented in the examples were performed with lithium acetate using Yeastmaker yeast transformation system (Clontech Laboratories, Palo Alto, Calif., USA).

### Preparation Example 1

### Cellulase Surface-Displaying Yeast

Below, a procedure of preparing a cellulase surface-displaying yeast (yeasts integrated so as to respectively surface-display endoglucanase and cellobiohydrolase, and β-glucosidase) will now be described.

### Preparation Example 1-1

### Preparation of Yeasts Provided with URA3, HIS3, TRP1, and LEU2 Auxotrophic Markers

### Preparation Example 1-1-1

### Provision of URA3 Marker with NBRC1440 Strain

A mutant URA3 fragment was obtained by PCR using a pair of primers of SEQ ID NOS: 1 and 2 with a genomic DNA commonly extracted from Saccharomyces cerevisiae MT8-1 (MATa ade his3 leu2 trp1 ura3) as a template. This fragment was transformed into Saccharomyces cerevisiae NBRC1440 (MATα), and a URA3 variant was selected in a 5-fluoroorotic acid (FOA) medium, giving an NBRC1440 strain provided with a URA3 marker.

The 5-fluoroorotic acid (FOA) medium was prepared in the following manner. A uracil dropout synthetic dextrose (SD) medium (Non-Patent Document 11) supplemented with 50 mg/L uracil acid and 2% (w/v) agar was autoclaved and kept at 65° C. FOA was dissolved in dimethyl sulfoxide (DMSO) to a concentration of 100 mg/mL and added to the autoclaved medium having a temperature of about 65° C. to a final FOA concentration of 1 mg/mL.

### Preparation Example 1-1-2

### Provision of HIS3 Marker with NBRC1440 Strain

Fusion PCR was carried out as follows:

In PCR1, a pair of HIS3-Green U (SEQ ID NO: 3; Forward) and HIS3-Green R (SEQ ID NO: 4; Reverse) primers were used with a genomic DNA commonly extracted from Saccharomyces cerevisiae NBRC1440 as a template to prepare a sequence upstream of HIS3.

In PCR2, a pair of URA3 fragment (SEQ ID NO: 5; Forward) and HIS3-40Uc (SEQ ID NO: 6; Reverse) primers were used with a pRS406 plasmid (Stratagene) as a template to prepare URA3.

In PCR3, a pair of HIS3-Green U (SEQ ID NO: 3; Forward) and HIS3-40Uc (SEQ ID NO: 6; Reverse) primers were used with mixed products of PCR1 and PCR2 as a template to prepare a fused fragment.

Using the obtained fused fragment, the NBRC1440 strain provided with a URA3 marker prepared above was transformed by homologous recombination. A strain lacking a uracil auxotrophy was selected on a uracil dropout (uracil-free medium) plate. The integration of this construct into the chromosome of the foregoing industrial yeast NBRC1440 results in the disruption of the HIS3 gene and the integration of the URA3 marker and its flanking repeat sequences into the chromosome.

Thereafter, this transformant was grown in YPD medium at 30° C. for 24 hours. Next, the transformant was grown to 1.0×10⁷ cells/200 μL on 5-FOA medium plate. All of colonies grown on 5-FOA medium plate, which have a uracil auxotrophic (Ura⁻) phenotype, were selected. The transformant grown on 5-FOA medium plate represented a uracil auxotrophic (Ura⁻) phenotype, since the URA3 marker which should have been introduced by transformation was removed from the chromosome of the transformant by the homologous recombination with the flanking repeat sequences of the URA3 marker.

Eventually, a strain lacking the HIS3 gene and the URA3 gene and having HIS3 and URA3 auxotrophies, i.e., an NBRC1440 strain provided with URA3 and HIS3 markers was obtained.

### Preparation Example 1-1-3

### Provision of TRP1 Marker with NBRC1440 Strain

Fusion PCR was carried out as follows:

In PCR1, a pair of TRP1-988 (SEQ ID NO: 7; Forward) and RP1-28r (SEQ ID NO: 8; Reverse) primers were used with the genomic DNA of Saccharomyces cerevisiae NBRC1440 as a template to prepare a sequence upstream of TRP1.

In PCR2, a pair of TRP1-URA3 (SEQ ID NO: 9; Forward) and TRP1-40r (SEQ ID NO: 10; Reverse) primers were used with a pRS406 plasmid (Stratagene) as a template to prepare URA3.

In PCR3, a pair of TRP1-988 (SEQ ID NO: 7; Forward) and TRP1-40r (SEQ ID NO: 10; Reverse) primers were used with mixed products of PCR1 and PCR2 as a template to prepare a fused fragment.

Using this fused fragment, the NBRC1440 strain provided with HIS3 and URA3 markers prepared above was transformed in the same manner as in Preparation Example 1-1-2, and eventually an NBRC1440 strain provided with URA3, HIS3, and TRP1 markers was obtained.

### Preparation Example 1-1-4

### Provision of LEU2 Marker with NBRC1440 Strain

Fusion PCR was carried out as follows:

In PCR1, a pair of LEU2-UP 3rd (SEQ ID NO: 11; Forward) and LEU2-down 3rd (SEQ ID NO: 12; Reverse) primers were used with the genomic DNA of Saccharomyces cerevisiae NBRC1440 as a template to prepare a sequence upstream of LEU2.

In PCR2, a pair of LEU2-URA3 3rd (SEQ ID NO: 13; Forward) and LEU2-40r (SEQ ID NO: 14; Reverse) primers were used with a pRS406 plasmid (Stratagene) as a template to prepare URA3.

In PCR3, a pair of LEU2-UP 3rd (SEQ ID NO: 11; Forward) and LEU2-40r (SEQ ID NO: 14; Reverse) primers were used with mixed products of PCR1 and PCR2 as a template to prepare a fused fragment.

Using this fused fragment, the NBRC1440 strain provided with URA3, HIS3, and TRP1, and LEU2 markers prepared above was transformed in the same manner as in Preparation Example 1-1-2, and eventually an NBRC1440 strain provided with URA3, HIS3, and TRP1, and LEU2 markers was obtained. This strain is denoted “NBRC1440/UHWL” for convenience.

### Preparation Example 1-2

### Preparation of pRS406 EG CBH2

First, a plasmid pGK406 EG, having a uracil gene (URA3) marker and for the surface-display integration of a gene for Trichoderma reesei-derived endoglucanase II (EGII), was constructed.

A 2719 bp DNA fragment containing the secretion signal sequence of Rhizopus oryzae-derived glucoamylase gene, an EGII gene, and the 3′ half of α-agglutinin gene (Non-Patent Document 12) was prepared by PCR using a pair of primers of SEQ ID NO: 15 (Forward) and SEQ ID NO: 16 (Reverse) with pEG23u31H6 (Non-Patent Document 13) as a template.

Two DNA fragments of PGK (phosphoglycerate kinase) promoter and PGK terminator were prepared by PCR using a primer pair of SEQ ID NO: 17 (Forward) and SEQ ID NO: 18 (Reverse) and a primer pair of SEQ ID NO: 19 (Forward) and SEQ ID NO: 20 (Reverse) designed for PGK promoter and PGK terminator, respectively, with a genomic DNA commonly extracted from Saccharomyces cerevisiae BY4741, obtained by American Type Culture Collection (ATCC), as a template. A multicloning site was prepared by PCR using a pair of primers of SEQ ID NO: 21 (Forward) and SEQ ID NO: 22 (Reverse). The PGK promoter was digested with XhoI and NheI, the multicloning site was digested with NheI and BglII, and the PGK terminator was digested with BglII and NotI, and then they were cloned into the XhoI-NotI site of pTA2 vector (Toyobo Co. Ltd.). The resultant vector was digested with XhoI and NotI, and the resultant fragment was cloned in pRS406 (Stratagene), thus giving a vector which was named pGK406.

The foregoing 2719 bp DNA fragment was digested with NheI and XmaI and inserted between the NheI site and the XmaI site of the plasmid pGK406 containing a URA3 gene and its promoter and terminator, a PGK promoter, and a PGK terminator, thus giving a plasmid containing a URA3 gene and its promoter and terminator, a PGK promoter, the secretion signal sequence of Rhizopus oryzae-derived glucoamylase gene, an endoglucanase (EGII) gene, the 3′ half of α-agglutinin gene, and a PGK terminator. The resultant plasmid was named pGK406 EG.

A fragment containing a GAPDH (glyceraldehyde triphosphate dehydrogenase) promoter, the secretion signal sequence of Rhizopus oryzae-derived glucoamylase gene, a Trichoderma reesei-derived CBH2 gene, the 3′ half of α-agglutinin gene, and a GAPDH terminator was prepared by PCR using a pair of primers of SEQ ID NO: 23 (Forward) and SEQ ID NO: 24 (Reverse) with a plasmid pFCBH2w3 (Non-Patent Document 14) as a template. The resultant fragment was digested with NotI and cloned in pGK406 EG digested with NotI. The resultant plasmid was named pRS406 EG CBH2.

### Preparation Example 1-3

### Preparation of pRS403 EG CBH2

First, a plasmid pGK403 EG, having a histidine gene (HIS3) marker and for the surface-display integration of a gene for Trichoderma reesei-derived endoglucanase II (EGII), was constructed.

A fragment containing a PGK promoter, the secretion signal sequence of Rhizopus oryzae-derived glucoamylase gene, an EGII gene, and a PGK terminator was cut out from pGK406 using ApaI and NotI, and ligated into pRS403 (Stratagene) digested with ApaI and NotI. The resultant plasmid was named pGK403 EG.

A fragment containing a GAPDH (glyceraldehyde triphosphate dehydrogenase) promoter, the secretion signal sequence of Rhizopus oryzae-derived glucoamylase gene, a Trichoderma reesei-derived CBH2 gene, the 3′ half of α-agglutinin gene, and a GAPDH terminator was digested with NotI and ligated into pGK403 EG digested with NotI. The resultant plasmid was named pRS403 EG CBH2.

### Preparation Example 1-4

### Preparation of pRS405 EG CBH2

First, a plasmid pGK405 EG, having a leucine gene (LEU2) marker and for the surface-display integration of a gene for Trichoderma reesei-derived endoglucanase II (EGII), was constructed.

A fragment containing a PGK promoter, a Rhizopus oryzae-derived glucoamylase gene secretion signal sequence, an EGII gene, and a PGK terminator was cut out from pGK406 using ApaI and NotI, and ligated into pRS405 (Stratagene) digested with ApaI and NotI. The resultant plasmid was named pGK405 EG.

A fragment containing a GAPDH (glyceraldehyde triphosphate dehydrogenase) promoter, the secretion signal sequence of Rhizopus oryzae-derived glucoamylase gene, a Trichoderma reesei-derived CBH2 gene, the 3′ half of α-agglutinin gene, and a GAPDH terminator was digested with NotI and ligated into pGK405 EG digested with NotI. The resultant plasmid was named pRS405 EG CBH2.

### Preparation Example 1-5

### Preparation of pIWBGL

A 2.5 kbp NcoI-XhoI DNA fragment coding for an Aspergillus aculeatus-derived β-glucosidase 1 (BGL1) gene was prepared by PCR using a pair of bgll primer 1 (SEQ ID NO: 25; Forward) and bgl1 primer 2 (SEQ ID NO: 26; Reverse) with a plasmid pBG211 (donated by Kyoto University) as a template. This DNA fragment was digested with NcoI and XhoI and inserted into the NcoI-XhoI site of a cell surface expression plasmid pIHCS (Non-Patent Document 13) containing the secretion signal sequence of Rhizopus oryzae-derived glucoamylase gene and the 3′ half of α-agglutinin gene (Non-Patent Document 12). The resultant plasmid was named pIBG13.

With this pIBG13 as a template, PCR was carried out using a pair of primers of SEQ ID NO: 23 (Forward) and SEQ ID NO: 24 (Reverse), giving a fragment containing a GAPDH promoter, the secretion signal sequence of Rhizopus oryzae-derived glucoamylase, a BGL1 gene, the 3′ half of α-agglutinin gene, and a GAPDH terminator. This fragment was digested with NotI and ligated into pRS404 digested with NotI. The resultant plasmid was named pIWBGL.

### Preparation Example 1-6

### Preparation of Yeast Strain having Endoglucanase II and Cellobiohydrolase 2 Integrated Both in a Copy Number of 1

NBRC1440/UHWL was transformed with pRS406 EG CBH2 processed into a linear form by cleaving it with a restriction enzyme NdeI, and a strain lacking uracil auxotrophy was selected on uracil-dropout (uracil-free medium) plate. Gene introduction was confirmed by the restoration of the destroyed URA3 gene in NBRC1440/UHWL on the transformation with pRS406 EG CBH2. The resultant strain was named “NBRC1440/pRS406 EG CBH2”.

### Preparation Example 1-7

### Preparation of Yeast Strain having Endoglucanase II and Cellobiohydrolase 2 Integrated Both in a Copy Number of 2

NBRC1440/pRS406 EG CBH2 was transformed with pRS403 EG CBH2 processed into a linear form by cleaving it with a restriction enzyme NdeI, and a strain lacking histidine auxotrophy was selected on histidine dropout (histidine-free medium) plate. Gene introduction was confirmed by the restoration of the destroyed HIS3 gene in NBRC1440/pRS406 EG CBH2 on the transformation with pRS403 EG CBH2. The resultant strain was named “NBRC1440/pRS406 EG CBH2/pRS403 EG CBH2”.

### Preparation Example 1-8

### Preparation of Yeast Strain having Endoglucanase II and Cellobiohydrolase 2 Integrated Both in a Copy Number of 3

NBRC1440/pRS406 EG CBH2/pRS403 EG CBH2 was transformed having pRS405 EG CBH2 processed into a linear form by cleaving it with a restriction enzyme HpaI, and a strain lacking leucine auxotrophy was selected on leucine dropout (leucine-free medium) plate. Gene introduction was confirmed by the restoration of the destroyed LEU2 gene in NBRC1440/pRS406 EG CBH2/pRS403 EG CBH2 on the transformation with pRS405 EG CBH2. The resultant strain was named “NBRC1440/pRS406 EG CBH2/pRS403 EG CBH2/pRS405 EG CBH2” which may also be simply referred to as “NBRC1440/EG-CBH2-3c”.

### Preparation Example 1-9

### Integration of β-Glucosidase 1 Gene for Surface Display

NBRC1440/pRS406 EG CBH2/pRS403 EG CBH2/pRS405 EG CBH2 (NBRC1440/EG-CBH2-3c) was transformed with pIWBGL processed into a linear form by cleaving it with Bst1107I. A strain lacking tryptophan auxotrophy was selected on tryptophan dropout (tryptophan-free medium) plate. Introduction of the β-glucosidase 1 gene was confirmed by the restoration of the destroyed TRP1 gene in each strain on the transformation with pIWBGL. Thus, strains having endoglucanase II and cellobiohydrolase 2 both in a copy number of 3 to be surface-displayed and also β-glucosidase 1 to be surface-displayed were obtained. The resultant transformants were simply denoted “NBRC1440/EG-CBH2-3c/BGL”.

### Preparation Example 2

### Cellulase Secreting Yeast

Below, a procedure of preparing a yeast integrated to secrete endoglucanase and cellobiohydrolase but surface-display β-glucosidase (hereinafter, which may be also referred to as a “cellulase secreting yeast” for convenience) will now be described.

### Preparation Example 2-1

### Plasmid pRS403/ssEG2-CBH2

A plasmid pRS403/ssEG2-CBH2, having a histidine gene (HIS3) marker and for integrating endoglucanase II (EGII) and cellobiohydrolase 2 (CBH2) to secrete them, was constructed.

A DNA fragment encoding a GAPDH promoter, a multi-cloning site (SalI, XbaI, BamHI, SmaI, XmaI), and a GAPDH terminator was prepared by PCR using a pair of primers XYL2c-Xho(F) (SEQ ID NO: 27; Forward) and XYL2c-NotI(R) (SEQ ID NO: 28; Reverse) with pUGP3 (Non-Patent Document 15) as a template. The fragment was inserted at the XhoI/NotI site of pRS403 (Stratagene), thus giving a plasmid pIHGP3.

A 1308 bp DNA fragment containing the secretion signal sequence of Rhizopus oryzae-derived glucoamylase gene and a Trichoderma reesei-derived endoglucanase (EGII) gene was prepared by PCR using a pair of primers of SEQ ID NO: 29 (Forward) and SEQ ID NO: 30 (Reverse) with pEG23u31H6 as a template.

The foregoing 1308 bp DNA fragment was digested with SmaI and inserted at the SmaI site of the plasmid pIHGP3 containing a HIS3 gene and its promoter and terminator, a GAPDH promoter, and a GAPDH terminator, thus giving a plasmid containing a HIS3 gene and its promoter and terminator, a GAPDH promoter, the secretion signal sequence of glucoamylase gene, an EGII gene, and a GAPDH terminator. The resultant plasmid was named pRS403/ssEG2.

A 1416 bp DNA fragment coding for the secretion signal sequence of Rhizopus oryzae-derived glucoamylase gene and a Trichoderma reesei-derived CBH2 gene was prepared by PCR using a pair of primers of SEQ ID NO: 31 (Forward) and SEQ ID NO: 32 (Reverse) with a plasmid pFCBH2w3 as a template.

The foregoing 1416 bp DNA fragment was digested with SmaI and inserted at the SmaI site of the plasmid pIHGP3 containing a HIS3 gene and its promoter and terminator, a GAPDH promoter, and a GAPDH terminator, thus giving a plasmid containing a HIS3 gene and its promoter and terminator, a GAPDH promoter, the secretion signal sequence of glucoamylase gene, a CBH2 gene, and a GAPDH terminator. The resultant plasmid was named pRS403/ssCBH2.

A fragment containing a GAPDH (glyceraldehyde triphosphate dehydrogenase) promoter, the secretion signal sequence of Rhizopus oryzae-derived glucoamylase gene, a Trichoderma reesei-derived CBH2 gene, and a GAPDH terminator was digested with NotI and ligated into pRS403/ssEG2 digested with NotI. The resultant plasmid was named pRS403/ssEG2-CBH2.

### Preparation Example 2-2

### Plasmid pRS405/ssEG2-CBH2

A fragment containing a GAPDH promoter, the secretion signal sequence of Rhizopus oryzae-derived glucoamylase gene, a cellobiohydrolase (CBH2) gene, and a GAPDH terminator was prepared by digesting pRS403/ssCBH2 with ApaI and NotI.

The foregoing fragment was inserted into pRS405, having a LEU2 gene marker, which had been digested with ApaI and NotI. The resultant plasmid was named pRS405/ssCBH2.

A fragment prepared by PCR using a pair of primers of SEQ ID NO: 23 (Forward) and SEQ ID NO: 24 (Reverse) with pRS403/ssEG2 as a template was digested with NotI and ligated into pRS405/ssCBH2 digested with NotI. The resultant plasmid was named pRS405/ssEG2-CBH2.

### Preparation Example 2-3

### Plasmid pRS406/ssEG2-CBH2

A fragment containing a GAPDH promoter, the secretion signal sequence of Rhizopus oryzae-derived glucoamylase gene, a cellobiohydrolase 2 (CBH2) gene, and a GAPDH terminator was prepared by digesting pRS403/ssCBH2 with ApaI and NotI.

The foregoing fragment was inserted into pRS406, having an URA3 gene marker, which had been digested with ApaI and NotI. The resultant plasmid was named pRS406/ssCBH2.

A fragment prepared by PCR using a pair of primers of SEQ ID NO: 23 (Forward) and SEQ ID NO: 24 (Reverse) with pRS403/ssEG2 as a template was digested with NotI and ligated into pRS406/ssCBH2 digested with NotI. The resultant plasmid was named pRS406/ssEG2-CBH2.

### Preparation Example 2-4

### Preparation of Yeast Strain having Endoglucanase II and Cellobiohydrolase 2 Integrated Both in a Copy Number of 1 for Secretion

NBRC1440/UHWL (Preparation Example 1-1) was transformed with pRS406/ssEG2-CBH2 processed into a linear form by cleaving it with a restriction enzyme NdeI, and a strain lacking uracil auxotrophy was selected on uracil-dropout (uracil-free medium) plate. Gene introduction was confirmed by the restoration of the destroyed URA3 gene in NBRC1440/UHWL on the transformation with pRS406/ssEG2-CBH2. The resultant strain was named “NBRC1440/pRS406/ssEG2-CBH2”.

### Preparation Example 2-5

### Preparation of Yeast Strain having Endoglucanase II and Cellobiohydrolase 2 Integrated Both in a Copy Number of 2

NBRC1440/pRS406/ssEG2-CBH2 was transformed with pRS403/ssEG2-CBH2 processed into a linear form by cleaving it with a restriction enzyme NdeI, and a strain lacking histidine auxotrophy was selected on histidine-dropout (histidine-free medium) plate. Gene introduction was confirmed by the restoration of the destroyed HIS3 gene in NBRC1440/pRS406/ssEG2-CBH2 on the transformation with pRS403/ssEG2-CBH2. The resultant strain was named “NBRC1440/pRS406/ssEG2-CBH2/pRS403/ssEG2-CBH2”.

### Preparation Example 2-6

### Preparation of Yeast Strain having Endoglucanase II and Cellobiohydrolase 2 Integrated Both in a Copy Number of 3

NBRC1440/pRS406/ssEG2-CBH2/pRS403/ssEG2-CBH2 was transformed with pRS405 ssEG2-CBH2 processed into a linear form by cleaving it with a restriction enzyme HpaI, and a strain lacking leucine auxotrophy was selected on leucine-dropout (leucine-free medium) plate. Gene introduction was confirmed by the restoration of the destroyed LEU2 gene in NBRC1440/pRS406/ssEG2-CBH2/pRS403/ssEG2-CBH2 on the transformation with pRS405 ssEG2-CBH2. The resultant strain was named “NBRC1440/pRS406/ssEG2-CBH2/pRS403/ssEG2-CBH2/pRS405 ssEG2-CBH2” which may also be simply referred to as “NBRC1440/ss-EG-CBH2-3c”.

### Preparation Example 2-7

### Integration of β-Glucosidase 1 Gene for Surface Display

NBRC1440/pRS406/ssEG2-CBH2/pRS403/ssEG2-CBH2/pRS405 ssEG2-CBH2 (NBRC1440/ss-EG-CBH2-3c) was transformed with pIWBGL processed into a linear form by cleaving it with Bst1107I. A strain lacking tryptophan auxotrophy was selected on tryptophan dropout (tryptophan-free medium) plate. Introduction of the β-glucosidase 1 gene was confirmed by the restoration of the destroyed TRP1 gene on the transformation with pIWBGL. The resultant strain may be simply denoted “NBRC1440/ss-EG-CBH2-3c/BGL”. In this Preparation Example, a strain secreting endoglucanase II and cellobiohydrolase 2 both in a copy number of 3 as in Preparation Example 2-6 and having β-glucosidase 1 to be surface-displayed was created.

### Reference Example 1

### Fed-Batch Fermentation Using Hot-Water-Treated Corn Fiber

Powdered corn fiber was mixed with water so as to attain a concentration of about 10% by weight, and the mixture was introduced into the reactor of autoclave device (model SR) for hydrothermal hot-pressing (Shin Sakashita Seisakusho Company Limited) and treated at 180° C. for 30 minutes (which may also be simply referred to as a “hot-water treatment”), and then dried in a drier at 70° C. for 12 hours to remove moisture, thus giving solid matter. This solid matter was used as a fermentation substrate.

Cell pellets of a yeast were prepared in the following manner. A yeast was precultured aerobically (in a dissolved oxygen concentration of about 2.0 ppm) for 24 hours at about 30° C., a pH of about 5.0 in SD medium (synthetic dextrose medium: containing 6.7 g/L yeast nitrogen base without amino acids [Difco] and suitable supplements; 20 g/L glucose added as a single carbon source) supplemented with essential amino acids, and then cultured at 30° C. for 48 hours in YPD medium (yeast extract polypeptone dextrose medium: containing 10 g/L yeast extract, 20 g/L polypeptone, and 20 g/L glucose) under the same conditions. Culture supernatant and cell pellets were separated by centrifugation at 6,000×g for 10 minutes at 4° C., thus giving cell pellets. The cell concentration was adjusted so as to be 75 g/L (wet cells) at the beginning of fermentation.

Cell pellets of NBRC1440/EG-CBH2-3c/BGL (cellulase surface-displaying yeast) or NBRC1440 cells (wild-type yeast) were inoculated in a fermentation medium containing 50 g/L fermentation substrate, 10 g/L yeast extract, 20 g/L polypeptone, 50 mM citrate buffer (pH 5.0), and 0.5 g/L potassium disulfite. Subsequently, cells were cultured anaerobically (in a dissolved oxygen concentration of about 0.05 ppm) at about 30° C. to carry out fermentation. At the beginning of fermentation, 500 μL (3.8 FPU) of cellulase (Cellulase SS, Nagase ChemteX Corporation) was added. Fermentation was carried out for 72 hours.

The concentration of ethanol during fermentation was measured by HPLC using a refractive index (RI) detector (L-2490 RI detector, Hitachi, Ltd.) and Shim-pack SPR—Pb Column (Shimadzu Corporation) as a separation column with water at a flow rate of 0.6 mL/min as a mobile phase, which was operated at 80° C.

When the hot-water-treated corn fiber was used as a fermentation substrate, the fermentation medium grew viscous during the process of fermentation reaction. While the fermentation substrate was additionally input in an amount of 50 g/L each at the points of 12 hours, 24 hours, and 48 hours after the beginning of fermentation, such additional addition became more and more difficult. The effect of the cellulase surface-displaying yeast was not almost observed.

### Reference Example 2

### Fed-Batch Fermentation Using Ammonia-Treated Corn Fiber

Powdered corn fiber was mixed with water so as to attain a concentration of about 10% by weight, and this mixture were introduced with 15 wt % aqueous ammonia into the reactor of autoclave device (model SR) for hydrothermal hot-pressing (Shin Sakashita Seisakusho Company Limited) and treated at 170° C. for 45 minutes (which may also be simply referred to as an “ammonia treatment”). Subsequently, the temperature was lowered to 100° C. and the reactor of the hydrothermal hot-pressing device was then opened to evaporate moisture and ammonia. Subsequently, the residue was dried in the same manner as in Reference Example 1 to further remove moisture and ammonia, thus giving solid matter. This solid matter was used as a fermentation substrate.

Cell pellets of NBRC1440/EG-CBH2-3c/BGL (cellulase surface-displaying yeast) or NBRC1440 cells (wild-type yeast) prepared in the same manner as in Reference Example 1 (at a cell concentration at the beginning of fermentation of 75 g/L (wet cells)) were inoculated in a fermentation medium containing 50 g/L fermentation substrate, 10 g/L yeast extract, 20 g/L polypeptone, 50 mM citrate buffer (pH 5.0), and 0.5 g/L potassium disulfite, and then cultured in the same manner as in Reference Example 1 to carry out fermentation. At the beginning of fermentation, 500 μL (3.8 FPU) of cellulase was added. The concentration of ethanol during fermentation was measured in the same manner as in Reference Example 1.

When the hot-water-treated corn fiber was used as a fermentation substrate, it was observed that a larger amount of ethanol was produce with NBRC1440/EG-CBH2-3c/BGL (cellulase surface-displaying yeast) than with NBRC1440 (wild-type yeast). The fermentation medium grew viscous during the process of fermentation, making it difficult to perform stirring for additional input of the substrate. Accordingly, the amount per input was reduced and the frequency of input was increased such that the fermentation substrate was additionally input in an amount of 20 g/L each at the time points of 12 hours, 18 hours, 24 hours, 28 hours, 36 hours, and 48 hours after the beginning of fermentation. However, the fermentation medium grew viscous during the process of fermentation reaction even with the cellulase surface-displaying yeast, and the eventual viscosity of the fermentation medium was very high.

### Example 1

### Fed-Batch Fermentation Using Corn Fiber Subjected to Hot-Water Treatment Followed by Ammonia Treatment

In the same manner as in Reference Example 1, powdered corn fiber was subjected to a hot-water treatment and then dried to remove moisture, thus giving solid matter. Subsequently, in the same manner as in Reference Example 2, this solid matter was subjected to an ammonia treatment, and then evaporation and drying to remove moisture and ammonia, thus giving solid matter. This solid matter was used as a fermentation substrate.

Cell pellets of NBRC1440/EG-CBH2-3c/BGL (cellulase surface-displaying yeast) or NBRC1440 cells (wild-type yeast) prepared in the same manner as in Reference Example 1 (at a cell concentration at the beginning of fermentation was 75 g/L (wet cells)) were inoculated in a fermentation medium containing 50 g/L fermentation substrate, 10 g/L yeast extract, 20 g/L polypeptone, 50 mM citrate buffer (pH 5.0), and 0.5 g/L potassium disulfite, and then cultured in the same manner as in Reference Example 1 to carry out fermentation. At the beginning of fermentation, 500 μL (3.8 FPU) of cellulase was added. The concentration of ethanol during fermentation was measured in the same manner as in Reference Example 1.

When the corn fiber subjected to the hot-water treatment followed by the ammonia treatment was used as a fermentation substrate, the fermentation substrate was additionally input in an amount of 50 g/L each at the time points of 12 hours and 24 hours after the beginning of fermentation, and a large amount of ethanol was produced with the NBRC1440/EG-CBH2-3c/BGL strain (cellulase surface-displaying yeast) without additional input at the time point of 48 hours after the beginning of fermentation, at which the additional input was performed in Reference Examples 1 and 2. An increased viscosity of the fermentation medium immediately after input of the fermentation substrate was reduced during the process of fermentation, and the fermentation medium was watery at the end.

### Example 2

### Comparison of Cellulase Surface-Displaying Yeast and Cellulase Secreting Yeast for Ethanol Production by Fed-Batch Fermentation Using Corn Fiber Subjected to Hot-Water Treatment Followed by Ammonia Treatment

The fermentation substrate was prepared in the same manner as in Example 1.

Cell pellets of NBRC1440/EG-CBH2-3c/BGL (cellulase surface-displaying yeast: Preparation Example 1), NBRC1440/ss-EG-CBH2-3c/BGL (cellulase secreting yeast: Preparation Example 2), or NBRC1440 cells (wild-type yeast) prepared in the same manner as in Reference Example 1 (at a cell concentration at the beginning of fermentation of 75 g/L (wet cells)) were inoculated in a fermentation medium containing 50 g/L fermentation substrate, 10 g/L yeast extract, 20 g/L polypeptone, 50 mM citrate buffer (pH 5.0), and 0.5 g/L potassium disulfite, and then cultured in the same manner as in Reference Example 1 to carry out fermentation. The amount of cellulase added at the beginning of fermentation was 500 μL (3.8 FPU) of cellulase. The concentration of ethanol during fermentation was measured in the same manner as in Reference Example 1.

When the corn fiber subjected to the hot-water treatment followed by the ammonia treatment was used as a fermentation substrate, the fermentation substrate was additionally input in an amount of 50 g/L each at the time points of 12 hours and 24 hours after the beginning of fermentation. It was observed that the production of ethanol was enhanced with NBRC1440/EG-CBH2-3c/BGL (cellulase surface-displaying yeast) while the production of ethanol with NBRC1440/ss-EG-CBH2-3c/BGL (cellulase secreting yeast) was similar extent to that with NBRC1440 cells (wild-type yeast).

### Reference Example 3

### Fed-Batch Fermentation Using Hot-Water-Treated Rice Straw

In the same manner as in Reference Example 1, ground rice straw was subjected to a hot-water treatment and then dried, thus giving solid matter, except for that the hot-water treatment was carried out at 180° C. for 90 minutes. This solid matter was used as a fermentation substrate.

Cell pellets of NBRC1440/EG-CBH2-3c/BGL (cellulase surface-displaying yeast) or NBRC1440 cells (wild-type yeast) prepared in the same manner as in Reference Example 1 (at a cell concentration at the beginning of fermentation of 75 g/L (wet cells)) were inoculated in a fermentation medium containing 50 g/L fermentation substrate, 10 g/L yeast extract, 20 g/L polypeptone, 50 mM citrate buffer (pH 5.0), and 0.5 g/L potassium disulfite, and then cultured in the same manner as in Reference Example 1 to carry out fermentation. At the beginning of fermentation, 7.6 FPU/mL of cellulase (Cellulase SS, Nagase ChemteX Corporation) was added in an amount of 0.5 FPU/g substrate, i.e., 500 μL (3.8 FPU) of cellulase. The concentration of ethanol during fermentation was measured in the same manner as in Reference Example 1.

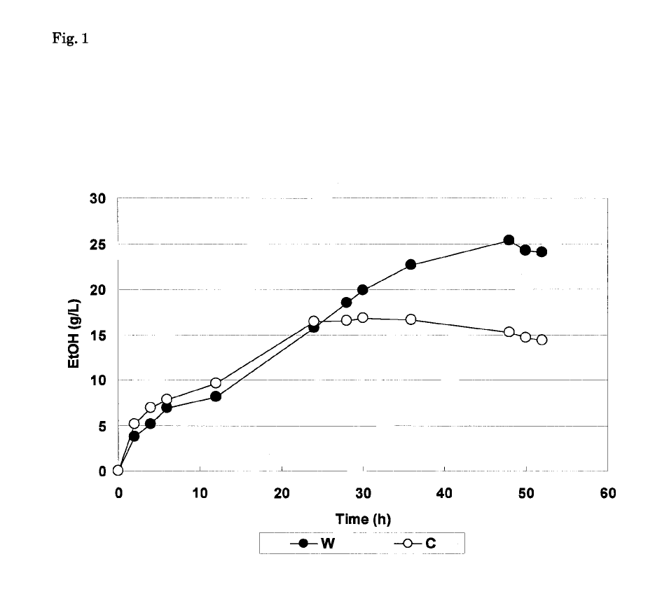

Results are shown in FIG. 1. FIG. 1 is a graph showing the time course of the production of ethanol by the cellulase surface-displaying yeast or the wild-type yeast with addition of cellulase in fed-batch fermentation using the hot-water-treated rice straw. In the graph, the horizontal axis indicates the fermentation time (hour), and the vertical axis indicates the produced amount of ethanol (g/L). In the figure, black circles indicate results of NBRC1440 (W: wild-type yeast), and white circles indicate results of NBRC1440/EG-CBH2-3c/BGL (C: cellulase surface-displaying yeast).

The fermentation substrate was additionally input in an amount of 40 g/L each at the time points of 12 hours, 24 hours, 36 hours, and 48 hours after the beginning of fermentation. Up to 24 hours after the beginning of fermentation, the production of ethanol was increased with NBRC1440/EG-CBH2-3c/BGL (cellulase surface-displaying yeast) as with the NBRC1440 cells (wild-type yeast), and the produced amount of ethanol was slightly higher with the cellulase surface-displaying yeast than with the wild-type yeast. However, after a lapse of 24 hours, while the production of ethanol continued to increase with the wild-type yeast, the production of of ethanol reached a plateau with the cellulase surface-displaying yeast. After a lapse of 48 hours, it was also observed that the production of ethanol reached a plateau with the wild-type yeast.

The generated amount of phenolic compounds during the foregoing fermentation was also investigated. The phenolic compounds may be generated from lignin of rice straw. The Folin-Ciocalteu assay was used to measure the amount of phenolic compounds.

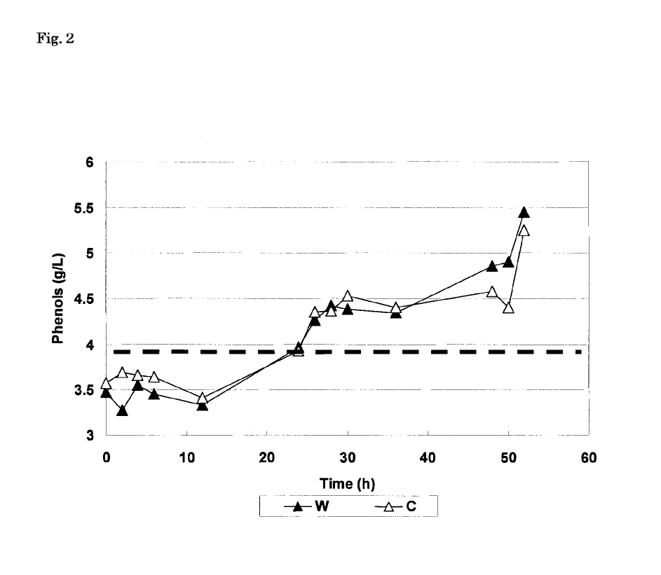

Results thereof are shown in FIG. 2. FIG. 2 is a graph showing the time course of the generation of phenolic compounds by the cellulase surface-displaying yeast or the wild-type yeast with addition of cellulase in fed-batch fermentation using the hot-water-treated rice straw. In the graph, the horizontal axis indicates the fermentation time (hour), and the vertical axis indicates the generated amount of phenolic compounds (g/L). In the figure, black triangles indicate results of NBRC1440 (W: wild-type yeast), and white triangles indicate results of NBRC1440/EG-CBH2-3c/BGL (C: cellulase surface-displaying yeast).

The generation of phenolic compounds was increased over time with either of the yeasts, and the generated amount was about 4 g/L at the time point of 24 hours after the beginning of fermentation, at which the production of ethanol reached a plateau with the cellulase surface-displaying yeast. It seems that as cellulose was monosaccharified during the degradation of rice straw by cellulase, lignin, which had been associated with cellulose, was eluted into the fermentation medium, leading to the generation of phenolic compounds. It was found that the production of ethanol with the cellulase surface-displaying yeast is affected by the generation of phenolic compounds from lignin in rice straw.

### Example 3

### Comparison of Ethanol Production by Fed-Batch Fermentation Using Hot-Water-Treated Rice Straw, Ammonia-Treated Rice Straw, and Rice Straw Subjected to Hot-Water Treatment Followed by Ammonia Treatment

In this example, hot-water-treated rice straw, ammonia-treated rice straw, and rice straw subjected to hot-water treatment followed by ammonia treatment were used as the respective fermentation substrates for carrying out fed-batch fermentation, respectively, and examined for the production of ethanol.

As for hot-water-treated rice straw, the hot-water-treated rice straw of Reference Example 3 was used. As for ammonia-treated rice straw, in the same manner as in Reference Example 2, ground rice straw was subjected to an ammonia treatment, and then evaporation and drying to remove moisture and ammonia, thus giving solid matter, except for that the ammonia treatment was carried out at 170° C. for 90 minutes. As for rice straw subjected to hot-water treatment followed by ammonia treatment, in the same manner as in Example 1, ground rice straw was subjected to a hot-water treatment and then subjected to an ammonia treatment, and then evaporation and drying to remove moisture and ammonia, thus giving solid matter, except for that the hot-water treatment was carried out at 180° C. for 90 minutes, and the ammonia treatment was carried out at 170° C. for 90 minutes.

Cell pellets of NBRC1440/EG-CBH2-3c/BGL (cellulase surface-displaying yeast: C) or NBRC1440 cells (wild-type yeast: W) prepared in the same manner as in Reference Example 1 (at a cell concentration at the beginning of fermentation was 75 g/L (wet cells)) were inoculated in a fermentation medium containing 50 g/L fermentation substrate, 10 g/L yeast extract, 20 g/L polypeptone, 50 mM citrate buffer (pH 5.0), and 0.5 g/L potassium disulfite, and then cultured in the same manner as in Reference Example 1 to carry out fermentation. At the beginning of fermentation, 7.6 FPU/mL of cellulase (Cellulase SS, Nagase ChemteX Corporation) was added in an amount of 0.5 FPU/g substrate, i.e., 500 μL (3.8 FPU) of cellulase. The ethanol concentration during fermentation was measured in the same manner as in Reference Example 1.

For all the pre-treated fermentation substrates, the initial input amount of fermentation substrate was 50 g/L. The timing and amount for additional input of fermentation substrate were determined with monitoring the viscosity of slurry of the fermentation medium. A highly viscous slurry makes it difficult to stir the substrate in the fermentation medium. Therefore, when the hot-water-treated rice straw was used, the fermentation substrate was additionally input in an amount of 40 g/L each at the time points of 12 hours, 24 hours, 36 hours, and 48 hours after the beginning of fermentation; when the ammonia-treated rice straw was used, the fermentation substrate was additionally introduced in an amount of 40 g/L each at the time points of 12 hours, 24 hours, and 48 hours after the beginning of fermentation; and when the rice straw subjected to the hot-water treatment followed by the ammonia treatment was used, the fermentation substrate was additionally input in an amount of 50 g/L each at the time points of 6 hours, 24 hours, and 48 hours after the beginning of fermentation.

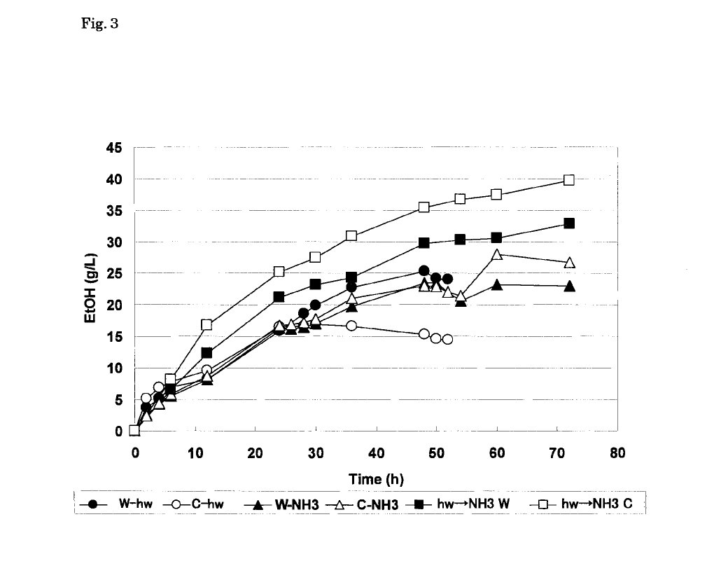

Results are shown in FIG. 3. FIG. 3 is a graph showing the time course of the production of ethanol by the cellulase surface-displaying yeast or the wild-type yeast with addition of cellulase in fed-batch fermentation using the hot-water-treated rice straw, the ammonia-treated rice straw, or the rice straw subjected to the hot-water treatment followed by the ammonia treatment. In the graph, the horizontal axis indicates the fermentation time (hour), and the vertical axis indicates the produced amount of ethanol (g/L). In the figure, black circles indicate results of NBRC1440+hot-water-treated rice straw (W-hw), white circles indicate results of NBRC1440/EG-CBH2-3c/BGL+hot-water-treated rice straw (C-hw), black triangles indicate results of NBRC1440+ammonia-treated rice straw (W—NH3), white triangles indicate results of NBRC1440/EG-CBH2-3c/BGL+ammonia-treated rice straw (C—NH3), black squares indicate results of NBRC1440+rice straw subjected to the hot-water treatment followed by the ammonia treatment (hw→NH3 W), and white squares indicate results of NBRC1440/EG-CBH2-3c/BGL+rice straw subjected to the hot-water treatment followed by the ammonia treatment (hw→NH3 C).

When the hot-water-treated rice straw was used, a similar observation was obtained to that in Reference Example 3. Specifically, up to 24 hours after the beginning of fermentation, the production of ethanol was increased with the cellulase surface-displaying yeast as with the wild-type yeast, and the produced amount of ethanol was slightly higher with the cellulase surface-displaying yeast than with the wild-type yeast. However, after a lapse of 24 hours, while the production of ethanol continued to increase with the wild-type yeast, the production of ethanol reached a plateau with the cellulase surface-displaying yeast. During the process of fermentation, the fermentation medium grew viscous.

When the ammonia-treated rice straw was used, the production of ethanol was increased with the cellulase surface-displaying yeast as with the wild-type yeast, and the eventually produced amount of ethanol was larger with the cellulase surface-displaying yeast than with the wild-type yeast. A plateau as observed using the hot-water-treated rice straw was not observed with the cellulase surface-displaying yeast. During the process of fermentation, the fermentation medium grew viscous, thus making it difficult to additionally input the fermentation substrate. The eventual viscosity of the fermentation medium was very high.

When the rice straw subjected to the hot-water treatment followed by the ammonia treatment was used, a larger amount of ethanol was produced with the cellulase surface-displaying yeast than with the wild-type yeast, and during the process of fermentation, the difference between the cellulase surface-displaying yeast and the wild-type yeast became significant. With either the cellulase surface-displaying yeast or the wild-type yeast, the viscosity of the fermentation medium was increased early after the input of the fermentation substrate, and subsequently reduced during the process of fermentation, and the fermentation medium was watery at the end. The viscosity was observed to be reduced faster with the cellulase surface-displaying yeast.

For both the cellulase surface-displaying yeast and the wild-type yeast, a larger amount of ethanol was produced using subjected to the hot-water treatment followed by the ammonia treatment than the two differently treated rice straws, and during the process of fermentation, the difference became significant. Among the respective conditions, the largest amount of ethanol was obtained by the fermentation with the cellulase surface-displaying yeast using the rice straw subjected to the hot-water treatment followed by the ammonia treatment as a fermentation substrate.

### Reference Example 4

### Fed-Batch Fermentation Using Hot-Water-Treated DDG

In the same manner as in Reference Example 1, DDG (Distiller's dried grain, a dried residue of corn distillation) was subjected to a hot-water treatment and then dried, thus giving solid matter. This solid matter was used as a fermentation substrate.

Cell pellets of a yeast were prepared in the following manner. A yeast was precultured aerobically (in a dissolved oxygen concentration of about 2.0 ppm) for 24 hours at about 30° C., a pH of about 5.0 in SD medium (synthetic dextrose medium: containing 6.7 g/L yeast nitrogen base without amino acids [Difco] and suitable supplements; 20 g/L glucose added as a single carbon source) supplemented with essential amino acids, and then cultured at 30° C. for 48 hours in YPD medium (yeast extract polypeptone dextrose medium: containing 10 g/L yeast extract, 20 g/L polypeptone, and 20 g/L glucose) under the same conditions. Culture supernatant and cell pellets were separated by centrifugation at 6,000×g for 10 minutes at 4° C., thus giving cell pellets. The cell concentration was adjusted so as to be 75 g/L (wet cells) at the beginning of fermentation.

Cell pellets of NBRC1440/EG-CBH2-3c/BGL (cellulase surface-displaying yeast), NBRC1440/ss-EG-CBH2-3c/BGL (cellulase secreting yeast), or NBRC1440 cells (wild-type yeast) prepared in the same manner as in Reference Example 1 were inoculated into a fermentation medium containing 50 g/L fermentation substrate, 10 g/L yeast extract, 20 g/L polypeptone, 50 mM citrate buffer (pH 5.0), and 0.5 g/L potassium disulfite, and then cultured anaerobically (in a dissolved oxygen concentration of about 0.05 ppm) at about 30° C. to carry out fermentation. At the beginning of fermentation, 500 μL (3.8 FPU) of cellulase (Cellulase SS, Nagase ChemteX Corporation) was added. Fermentation was carried out for 72 hours.

The concentration of ethanol during fermentation was measured by HPLC using a refractive index (RI) detector (L-2490 RI detector, Hitachi, Ltd.) and Shim-pack SPR—Pb Column (Shimadzu Corporation) as a separation column with water at a flow rate of 0.6 mL/min as a mobile phase, which was operated at 80° C.

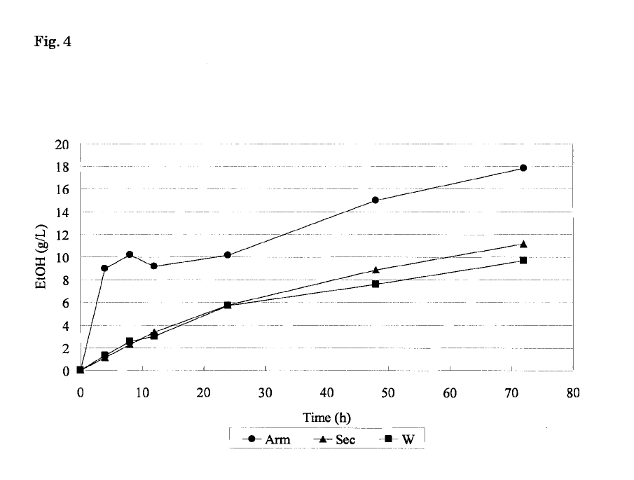

Results are shown in FIG. 4. FIG. 4 is a graph showing the time course of the production of ethanol by the cellulase surface-displaying yeast, the cellulase secreting yeast, or the wild-type yeast with addition of cellulase in fed-batch fermentation using the hot-water-treated DDG. The horizontal axis indicates the fermentation time (hour), and the vertical axis indicates the produced amount of ethanol (g/L). In the graph, black circles indicate results of NBRC1440/EG-CBH2-3c/BGL (Arm: cellulase surface-displaying yeast), black triangles indicate results of NBRC1440/ss-EG-CBH2-3c/BGL (Sec: cellulase secreting yeast), and black squares indicate results of NBRC1440 cells (W: wild-type yeast).

When the hot-water-treated DDG was used as a fermentation substrate, the fermentation reaction did not progress smoothly in fact. The fermentation substrate was additionally input in an amount of 50 g/L at the time point of 24 hours after the beginning of fermentation, but the yield of ethanol production did not readily increase even with the cellulase surface-displaying yeast. Also, with any of the yeasts, during the process of fermentation, the death of yeast was observed.

### Reference Example 5

### Fed-Batch Fermentation Using Ammonia-Treated DDG

In the same manner as in Reference Example 2, DDG was subjected to an ammonia treatment, and then evaporation and drying to remove moisture and ammonia, thus giving solid matter. This solid matter was used as a fermentation substrate.

Cell pellets of NBRC1440/EG-CBH2-3c/BGL (cellulase surface-displaying yeast), NBRC1440/ss-EG-CBH2-3c/BGL (cellulase secreting yeast), and NBRC1440 cells (wild-type yeast) prepared in the same manner as in Reference Example 1 (at a cell concentration at the beginning of fermentation of 75 g/L (wet cells)) were inoculated in a fermentation medium containing 50 g/L fermentation substrate, 10 g/L yeast extract, 20 g/L polypeptone, 50 mM citrate buffer (pH 5.0), and 0.5 g/L potassium disulfite, and then cultured in the same manner as in Reference Example 1 to carry out fermentation. At the beginning of fermentation, 500 μL (3.8 FPU) of cellulase was added. The concentration of ethanol during fermentation was measured in the same manner as in Reference Example 1.

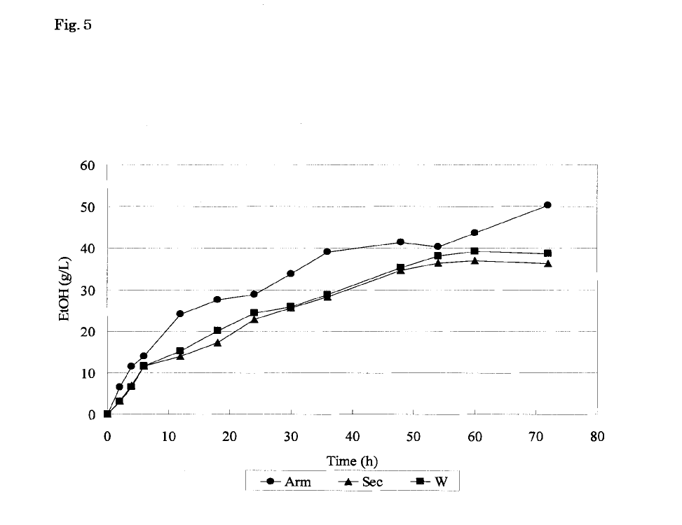

Results are shown in FIG. 5. FIG. 5 is a graph showing the time course of the production of ethanol by the cellulase surface-displaying yeast, the cellulase secreting yeast, or the wild-type yeast with addition of cellulase in fed-batch fermentation using the ammonia-treated DDG. The horizontal axis indicates the fermentation time (hour), and the vertical axis indicates the produced amount of ethanol (g/L). In the graph, black circles indicate results of NBRC1440/EG-CBH2-3c/BGL (Arm: cellulase surface-displaying yeast), black triangles indicate results of NBRC1440/ss-EG-CBH2-3c/BGL (Sec: cellulase secreting yeast), and black squares indicate results of NBRC1440 cells (W: wild-type yeast).

When the ammonia-treated DDG was used, the fermentation medium grew viscous during the process of fermentation, making it difficult to perform stirring for additional input of the substrate. The fermentation substrate was additionally input in an amount of 50 g/L each at the time points of 12 hours, 24 hours, and 48 hours after the beginning of fermentation. The fermentation medium grew viscous during the process of fermentation even with the cellulase surface-displaying yeast, and the eventual viscosity of the fermentation medium was very high.

### Example 4

### Fed-Batch Fermentation Using DDG Subjected to Hot-Water Treatment Followed by Ammonia Treatment

DDG was subjected to a hot-water treatment and then an ammonia treatment in the same manner as in Example 1, thus giving solid matter. This solid matter was used as a fermentation substrate.

Cell pellets of NBRC1440/EG-CBH2-3c/BGL (cellulase surface-displaying yeast), NBRC1440/ss-EG-CBH2-3c/BGL (cellulase secreting yeast), or NBRC1440 cells (wild-type yeast) prepared in the same manner as in Reference Example 1 (at a cell concentration at the beginning of fermentation of 75 g/L (wet cells)) were inoculated in a fermentation medium containing 50 g/L fermentation substrate, 10 g/L yeast extract, 20 g/L polypeptone, 50 mM citrate buffer (pH 5.0), and 0.5 g/L potassium disulfite, and then cultured in the same manner as in Reference Example 1 to carry out fermentation. At the beginning of fermentation, 500 μL (3.8 FPU) of cellulase was added. The concentration of ethanol during fermentation was measured in the same manner as in Reference Example 1.

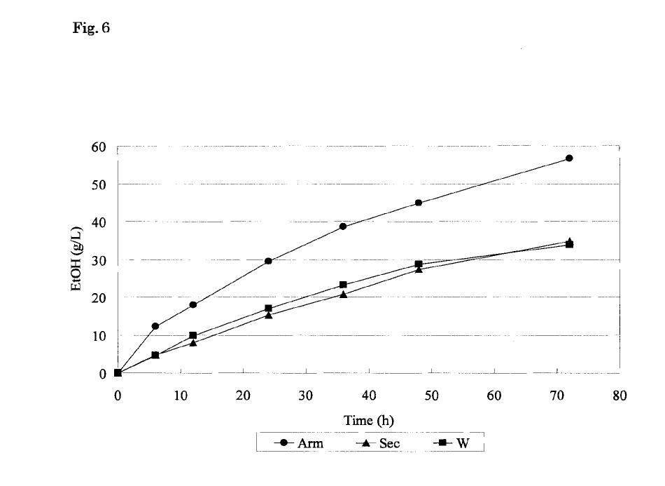

Results are shown in FIG. 6. FIG. 6 is a graph showing the time course of the production of ethanol by the cellulase surface-displaying yeast, the cellulase secreting yeast, or the wild-type yeast with addition of cellulase in fed-batch fermentation using the DDG subjected to the hot-water treatment followed by the ammonia treatment. The horizontal axis indicates the fermentation time (hour), and the vertical axis indicates the produced amount of ethanol (g/L). In the graph, black circles indicate results of NBRC1440/EG-CBH2-3c/BGL (Arm: cellulase surface-displaying yeast), black triangles indicate results of NBRC1440/ss-EG-CBH2-3c/BGL (Sec: cellulase secreting yeast), and black squares indicate results of NBRC1440 cells (W: wild-type yeast).

When the DDG subjected to the hot-water treatment followed by the ammonia treatment was used, the fermentation substrate was additionally input in an amount of 50 g/L each at the time points of 12 hours and 24 hours after the beginning of fermentation, and a large amount of ethanol was produced with the NBRC1440/EG-CBH2-3c/BGL strain (cellulase surface-displaying yeast) without additional input at the time point of 48 hours after the beginning of fermentation, at which the additional input was carried out in Reference Example 5. An increased viscosity of the fermentation medium immediately after the input of the fermentation substrate was reduced during the process of fermentation, and the fermentation medium was watery at the end.

For the DDG subjected to the hot-water treatment followed by the ammonia treatment, it was observed that the production of ethanol was enhanced with NBRC1440/EG-CBH2-3c/BGL (cellulase surface-displaying yeast) while the production of ethanol with NBRC1440/ss-EG-CBH2-3c/BGL (cellulase secreting yeast) was similar extent to that with the NBRC1440 cells (wild-type yeast).

No matter which substrate was used, when, prior to fermentation, the substrate was subjected to a hot-water treatment followed by an ammonia treatment, enhancement of ethanol production was achieved with no high viscosity of the fermentation medium during the process of fermentation. In particular, with the cellulase surface-displaying yeast, the ethanol production was enhanced.

### Example 5

### Fed-Batch Fermentation Using DDG Subjected to Hot-Water Treatment Followed by Ammonia Treatment or Subjected to Ammonia Treatment Followed by Hot-Water Treatment

DDG was subjected to a hot-water treatment (at 180° C. for 30 minutes) and then an ammonia treatment (at 170° C. for 45 minutes) in the same manner as in Example 1, thus giving solid matter, and this solid matter was used as a fermentation substrate subjected to hot-water treatment followed by ammonia treatment.

In the same manner as in Reference Example 2, DDG was subjected to an ammonia treatment (at 170° C. for 45 minutes), and then evaporation and drying to remove moisture and ammonia, thus giving solid matter, and in the same manner as in Reference Example 1 this solid matter was then subjected to a hot-water treatment (at 180° C. for 30 minutes) and then dried to remove moisture, thus giving solid matter, and this solid matter was used as a fermentation substrate subjected to ammonia treatment followed by hot-water treatment.

Cell pellets of NBRC1440/EG-CBH2-3c/BGL (cellulase surface-displaying yeast) or NBRC1440 cells (wild-type yeast) prepared in the same manner as in Reference Example 1 were inoculated in a fermentation medium containing 50 g/L fermentation substrate, 10 g/L yeast extract, 20 g/L polypeptone, 50 mM citrate buffer (pH 5.0), and 0.5 g/L potassium disulfite, and then cultured anaerobically (in a dissolved oxygen concentration of about 0.05 ppm) at about 30° C. to carry out fermentation. At the beginning of fermentation, 500 μL (3.8 FPU) of cellulase (Cellulase SS, Nagase ChemteX Corporation) was added. Fermentation was carried out for 72 hours. The concentration of ethanol during fermentation was measured in the same manner as in Reference Example 1.

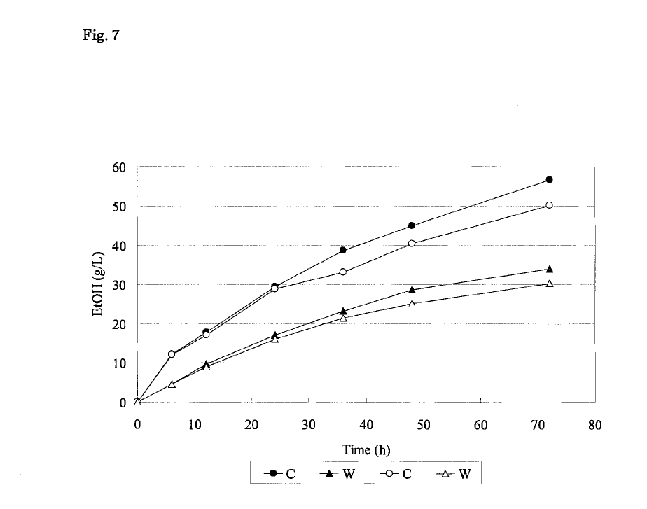

Results are shown in FIG. 7. FIG. 7 is a graph showing the time course of the production of ethanol by the cellulase surface-displaying yeast or the wild-type yeast with addition of cellulase in fed-batch fermentation using the DDG subjected to hot-water treatment followed by ammonia treatment or the DDG subjected to ammonia treatment followed by hot-water treatment. The horizontal axis indicates the fermentation time (hour), and the vertical axis indicates the produced amount of ethanol (g/L). Black circles indicate results of NBRC1440/EG-CBH2-3c/BGL (C: cellulase surface-displaying yeast) with the fermentation substrate subjected to hot-water treatment followed by ammonia treatment, black triangles indicate results of NBRC1440 cells (W: wild-type yeast) with the fermentation substrate subjected to hot-water treatment followed by ammonia treatment, white circles indicate results of NBRC1440/EG-CBH2-3c/BGL (C: cellulase surface-displaying yeast) with the fermentation substrate subjected to ammonia treatment followed by hot-water treatment, and white triangles indicate results of NBRC1440 cells (W: wild-type yeast) with the fermentation substrate subjected to ammonia treatment followed by hot-water treatment.

For either the fermentation substrate subjected to hot-water treatment followed by ammonia treatment or the fermentation substrate subjected to ammonia treatment followed by hot-water treatment, the fermentation substrate was additionally input in an amount of 50 g/L each at the time points of 12 hours and 24 hours after the beginning of fermentation. It was also found that using the fermentation substrate subjected to ammonia treatment followed by hot-water treatment, a large amount of ethanol was produced with NBRC1440/EG-CBH2-3c/BGL (cellulase surface-displaying yeast); and an increased viscosity of the fermentation medium immediately after the input of the fermentation substrate was reduced during the process of fermentation, and the fermentation medium was watery at the end. Comparing the pre-treatments, a larger amount of ethanol was produced with the fermentation substrate subjected to hot-water treatment followed by ammonia treatment than with the fermentation substrate subjected to ammonia treatment followed by hot-water treatment.

### INDUSTRIAL APPLICABILITY

According to the present invention, even when a cellulose-based material that contains lignin is used as a source material, enhancement of ethanol production in terms of the amount and rate can be achieved, making it advantageous to industrial production of ethanol. Also, corn fiber or DDG, which may be a by-product of the process of producing ethanol from corn, can be efficiently reused for ethanol production. The used amount of cellulase enzyme can be reduced. Ethanol can be efficiently produced from soft biomass, which may usually be disposed of, leading to the reduction of cost.

1. A method for producing ethanol, comprising:
subjecting a cellulose-based material to a hot-water treatment and an ammonia treatment to obtain a fermentation substrate; and
reacting the fermentation substrate with a yeast to produce ethanol.

2. The method according to claim 1, wherein the cellulose-based material is subjected to the hot-water treatment followed by the ammonia treatment.

3. The method according to claim 1, wherein the yeast is a cellulase yeast.

4. The method according to claim 3, wherein the cellulase yeast is a cellulase surface-displaying yeast that displays endoglucanase, cellobiohydrolase, and β-glucosidase on the cell surface.

5. The method according to claim 1, wherein the reacting is carried out by fed-batch addition of the fermentation substrate.

6. A method for producing a fermentation substrate for ethanol production by a yeast, comprising:
subjecting a cellulose material to a hot-water treatment and an ammonia treatment to obtain a fermentation substrate.

7. The method according to claim 2, wherein the yeast is a cellulase yeast.

8. The method according to claim 2, wherein the reacting is carried out by fed-batch addition of the fermentation substrate.

9. The method according to claim 3, wherein the reacting is carried out by fed-batch addition of the fermentation substrate.

10. The method according to claim 4, wherein the reacting is carried out by fed-batch addition of the fermentation substrate.

11. The method according to claim 7, wherein the cellulase yeast is a cellulase surface-displaying yeast that displays endoglucanase, cellobiohydrolase, and β-glucosidase on the cell surface.

12. The method according to claim 7, wherein the reacting is carried out by fed-batch addition of the fermentation substrate.

13. The method according to claim 11, wherein the reacting is carried out by fed-batch addition of the fermentation substrate.
